(19) United States
(12) Patent Application Publication
(10) Pub. No.: US 2004/0010045 A1
(43) Pub. Date: Jan. 15, 2004

(54) Therapeutic compositions comprised of pentamidine and methods of using same to treat cancer

(76) Inventor: Taolin Yi, Solon, OH (US)

Correspondence Address: Barbara E. Arndt, Ph.D., Jones Day, North Point, 901 Lakeside Avenue, Cleveland, OH, 44114, US

(21) Appl. No.: 10/427,887

(22) Filed: May 1, 2003

Related U.S. Application Data
(63) Continuation-in-part of application No. 10/354,357, filed on Jan. 30, 2003.
(63) Continuation-in-part of application No. 10/238,007, filed on Sep. 9, 2002.
(63) Continuation-in-part of application No. 10/238,007, filed on Sep. 9, 2002.
(60) Provisional application No. 60/460,735, filed on Apr. 4, 2003.
(60) Provisional application No. 60/376,789, filed on May 1, 2002.
(60) Provisional application No. 60/353,019, filed on Jan. 30, 2002.
(60) Provisional application No. 60/317,993, filed on Sep. 7, 2001.

(51) Int. Cl. A61K 31/155

(52) U.S. Cl. 514/631

(57) ABSTRACT

Pentamidine, an anti-protozoa drug, is described herein as a potent PTPase inhibitor with anti-cancer activity. Pentamidine at its therapeutic doses inhibits recombinant PRL phosphatases and inactivates intracellular PRLs in NIH3T3 transfectants. Pentamidine treatment at a nontoxic dose markedly inhibits the growth of WM9 human melanoma tumors in nude mice coincident with tumor cell necrosis and is capable of inactivating an ectopically expressed PRL-2 in the cancer cells. The drug has growth inhibitory activity against different human cancer cell lines that express the PRLs, and therefore has broad anti-cancer activity based on inactivating the oncogenic phosphatases.

This application claims the benefit of U.S. Provisional Application Serial No. 60/460,735, filed Apr. 4, 2003, and U.S. Provisional Application Serial No. 60/376,789, filed May 1, 2002. This application is a continuation-in-part of U.S. patent application Ser. No. 10/354,357, filed Jan. 30, 2003, which claims the benefit of U.S. Provisional Application Serial No. 60/353,019, filed Jan. 30, 2002, and which is a continuation-in-part of U.S. patent application, Ser. No. 10/238,007, filed Sep. 9, 2002, which claims the benefit of U.S. Provisional Application Serial No. 60/317,993, filed Sep. 7, 2001. The present application is also a continuation-in-part of said U.S. patent application, Ser. No. 10/238,007. All of the foregoing disclosures are hereby incorporated by reference in their entirety.

The U.S. Government has a paid-up license in this invention and the right in limited circumstances to require the patent owner to license others on reasonable terms as provided for by the terms of grant numbers R01CA79891 and R01MG58893 from the National Institutes of Health.

### BACKGROUND OF THE INVENTION

Protein tyrosine phosphorylation and dephosphorylation, catalyzed by protein tyrosine kinases (PTKs) and protein tyrosine phosphatases (PTPases), respectively, are key switches in many important eukaryotic cellular signaling pathways. The actions of PTKs and PTPases are in a state of dynamic equilibrium that determines the status of cellular protein tyrosine phosphorylation and plays a crucial role in the regulation of cell proliferation, differentiation, viability and functional activation.

PTPases comprise a large superfamily. Approximately 50 mammalian PTPases have been reported, with their total number in the human genome estimated to be about 100. PTPases can be subdivided into two major families based on their differential substrate specificity and distinct signature motifs conserved in their catalytic domains. Standard specificity PTPases, such as Src-homology protein tyrosine phosphatase 1 domain (SHP-1) and the like, exclusively dephosphorylate phosphotyrosine residues in protein substrates. In contrast, dual specificity phosphatases, such as mitogen-activated protein kinase phosphatase-1 (MKP-1) and the like, dephosphorylate phosphotyrosine residues as well as phosphoserine and phosphothreonine residues.

Despite their sequence homology and a common catalytic mechanism, PTPases play diverse roles in intracellular signaling and most act as negative signaling regulators. For example, SHP-1 phosphatase is a negative regulator of cytokine signaling via dephosphorylating and inactivating Jak/Stat proteins that mediate cytokine initiated signaling cascade 7. Similarly, PTP1B was shown to dephosphorylate Jak PTKs and thus may down regulate cytokine signaling mediated via the kinases.

A limited number of PTPases have been found to be oncogenic signaling molecules. For example, elevated expression of the PRL family of phosphatases such as, but not limited to, PRL-1, PRL-2, PRL-3, and the like, has been shown to have oncogenic effects in several experimental systems and may play a causitive role in human malignancies. The PRL phosphatases are also sometimes termed PTP (CAAX) or PTP4A. Several lines of evidence have demonstrated the oncogenic activity of the PRL phosphatase family. For example, ectopic expression of PRL PTPases has been found to enhance cell growth, cause cell transformation and/or promote tumor growth in nude mice. PRL-2 over-expression was detected in both androgen-dependent and androgen-independent prostate cancer cell lines and prostate tumor tissue. Recently, over-expression of PRL-3, as a result of gene amplification or other defects, was found to associate with tumor metastasis of human colorectal cancer. However, prior to this recent discovery, studies of PRL activity had been limited and the oncogenic mechanism of these PTPases remains undefined.

A gene coding for PRL-1 (phosphatase of regenerating liver-1) was identified as one of the genes expressed during liver regeneration. PRL-2 and PRL-3 were found more recently through searching sequence databases based on their homology to PRL-1. The three PRLs have been found to be closely related phosphatases with at least 75% amino acid sequence similarity. Among normal adult tissues, PRLs are expressed predominantly in skeletal muscle with lower expression levels detectable in brain (PRL-1), liver (PRL-2), heart (PRL-3) and pancreas (PRL-3). Although the physiologic functions of the PRLs are not yet identified, involvement of PRL-1 in cell proliferation has been suggested by its increased expression in regenerating liver. A role for PRLs in maintenance of differentiating epithelial tissues was proposed based on their selective expression in terminally differentiated cells in kidney and lung (PRL-1), as well as mouse intestine (PRL-3). The potential involvement of PRL-3 over-expression in other human malignancies is indicated by the localization of the PRL-3 gene at human chromosome 8 q and by the observation that extra copies of this region are often found in advanced stages of different tumor types. Although the PRLs have been shown to dephosphorylate synthetic tyrosine substrates in vitro, their in vivo substrates and substrate-specificity remain to be defined.

In addition to PRLs, PTPalpha has also been shown to be an oncogenic phosphatase. Similarly to PRLs, over-expression of PTPalpha caused cell transformation in vitro and tumorigenesis in a mouse model. Increased expression of this phosphatase was detected in human colon carcinoma and oral squamous cell carcinoma. Over-expression of the dual specificity cdc25 phosphatases (cdc25A and cdc25B) has also been detected in certain human malignancies.

Given the critical role of PTPases in intracellular signaling, inhibitors of the phosphatases might be expected to have therapeutic value. However, few clinically useful inhibitors of PTPases have been reported despite extensive efforts in the last decade to identify them. Small chemical inhibitors identified so far (e.g., sodium orthovanadate, pervanadate, sodium molybdic acid, iodione acetic acid, and the like) broadly inhibit all PTPases and thus are highly toxic. A number of peptide inhibitors have been reported but are excluded from clinical use because their large size prevents efficient intracellular delivery.

The oncogenic PRL family of phosphatases is an attractive target for developing inhibitors as anti-cancer therapeutics, given a potentially pathogenic role of PRL over-expression in human malignancies. Thus, identification of small synthetic compounds with specific PTPase inhibitory activity could result in therapeutic compositions that are useful for treating and/or preventing cancer and/or other pathological conditions associated with PRL phosphatase activity.

### SUMMARY OF THE INVENTION

Unexpectedly, it has been discovered that pentamidine, an anti-protozoa drug with an unknown mechanism of action, is a potent PTPase inhibitor with anti-cancer activity at therapeutic anti-protozoan dosages. In particular, it has been discovered that pentamidine inhibits recombinant PRL phosphatases and inactivates intracellular PRLs in NIH3T3 transfectants with an effective duration greater than 24 hours following a five minute pulse treatment of the cells. In addition, pentamidine has in vitro growth-inhibitory activity against human cancer cell lines that express the endogenous PRLs. It has further been discovered that pentamidine treatment, at a nontoxic dose, markedly inhibits the growth of WM9 human melanoma tumors in nude mice, coincident with tumor cell necrosis, and is capable of inactivating an ectopically expressed PRL-2 in the cancer cells. The drug has growth inhibitory activity against a broad range of human cancer cell lines that express PRL phosphatase, suggesting a broad anti-cancer activity based on inactivating the oncogenic phosphatases. It has also been unexpectedly discovered that pentamidine also is a potent inhibitor of PTP1B phosphatase activity in vitro.

In one embodiment of the invention, a therapeutic composition for preventing, treating or ameliorating cancer, preferably human cancer, comprises pentamidine, or a biological equivalent or derivative thereof. The term “pentamidine” is hereinafter meant to encompass all present and future biological equivalents and derivatives thereof. The pentamidine is preferably employed in an amount effective to inhibit phosphatase activity in cancer cells. Preferably, the pentamidine inhibits PRL phosphatases including, but not limited to, PRL-1, PRL-2, PRL-3, and combinations thereof. Preferably, the pentamidine inhibits PTP1B phosphatases. The therapeutic composition is envisioned for use in cancers such as, but not limited to, lymphoma, multiple myeloma, colon cancer, neuroblastoma, glioma, leukemia, melanoma, prostate cancer, breast cancer, renal cancer, bladder cancer, and the like.

The invention methods and compositions are not intended to be limited to the use of pentamidine in preventing, treating or ameliorating cancer, but they are further intended to encompass the use of pentamidine in preventing, treating or ameliorating any mammalian disease having an etiology related to cellular phosphatase activity.

Therefore, in another embodiment, a therapeutic composition for preventing, treating or ameliorating a mammalian disease, preferably a human disease, having an etiology related to cellular phosphatase activity, comprises pentamidine, or a biological equivalent or derivative thereof. Preferably, the pentamidine is present in an amount effective to inhibit phosphatase activity in the cells. In one embodiment, the pentamidine inhibits PTP1B phosphatase, the PRL phosphatases, and combinations of these.

In other embodiments of the invention, methods provided for preventing, treating or ameliorating cancer and/or a mammalian disease having an etiology related to cellular phosphatase activity, by administering to a mammal pentamidine, or a biological equivalent or derivative thereof. The methods are preferably useful for treating human cancer and/or other of the foregoing human diseases. Preferably, the pentamidine is administered in an amount effective to inhibit phosphatase activity in cancer cells and/or in the cells of other of the foregoing mammalian diseases. More preferably, the amount of pentamidine is effective to inhibit PRL phosphatases and/or PTP1B phosphatase.

In another embodiment, a method for preventing, treating or ameliorating cancer comprises administering an effective amount of a therapeutic composition comprising an agent, preferably pentamidine, that selectively inhibits a PRL phosphatase.

Embodiments of the invention are also provided as methods for identifying pentamidine-resistant or pentamidine-sensitive cancer cells, comprising isolating a PRL phosphatase from a cancer cell sample; and determining an amino acid sequence of the isolated PRL phosphatase, wherein the presence of a mutant amino acid sequence indicates pentamidine resistant cancer cells and the absence of a mutant amino acid sequence indicates pentamidine-sensitive cancer cells. A method for determining a risk for pentamidine-resistance or pentamidine-sensitivity is also provided, comprising isolating PRL phosphatase from a cancer cell sample; and determining an amino acid sequence of the isolated PRL phosphatase, wherein the presence of a mutant amino acid sequence indicates a risk for pentamidine resistance and the absence of a mutant amino acid sequence indicates pentamidine sensitivity.

Other embodiments are provided as methods for identifying pentamidine-resistant or pentamidine-sensitive cancer cells, comprising isolating a PRL phosphatase from a cancer cell sample; and testing the isolated phosphatase for phosphatase activity in the presence and absence of pentamidine, or a biological equivalent or derivative thereof, wherein inhibition of the phosphatase activity is indicative of pentamidine-sensitive cancer cells, and lack of inhibition of the phosphatase activity is indicative of pentamidine-resistant cancer cells. In another such embodiment, a method for identifying pentamidine-resistant or pentamidine-sensitive cancer cells, comprises isolating a cancer cell sample; and performing a cell growth assay to determine the growth of the cancer cells in the presence and absence of pentamidine, or a biological equivalent or derivative thereof, wherein inhibition of the cell growth is indicative of pentamidine-sensitive cancer cells, and lack of inhibition of the cell growth is indicative of pentamidine-resistant cancer cells.

Embodiments of the invention encompass mutant PRL phosphatases produced by in vitro substitution of one or more amino acid residues of wild-type PRL phosphatases. In some embodiments, the phosphatase activity of the mutant is not inhibited by pentamidine or a biological equivalent thereof. In other embodiments, the phosphatase activity of the mutant is not inhibited by a derivative of pentamidine. The invention embodiments further encompass mutant PRL phosphatases having SEQ ID NO: 4, SEQ ID NO: 5 and SEQ ID NO: 6.

### BRIEF DESCRIPTION OF THE DRAWINGS

FIG. 1 illustrates differential inhibitory activities of pentamidine against recombinant PTPases in vitro. Activities of GST (Glutathione S Transferase) fusion proteins of PTP1B (A), SHP-1 (D), SHP-2 (E), MKP1 (F) in dephosphorylating a phosphotyrosine peptide in the absence or presence of various amounts of PE or SSG (sodium stibogluconate) were measured in in vitro PTPase assays. Relative activities of GST-PTP1B fusion protein pre-incubated with PE (1 μg/ml) or SSG (10 μg/ml) and then washed (+) or not washed (−) were determined using the peptide substrate (B). Activities of GST/PTP1B fusion protein in dephosphorylating DiFMUP in the absence or presence of PE or SSG were also determined by PTPase assays (C). Data represent mean±s.d. values of triplicate samples.

FIG. 2 illustrates that pentamidine inhibits recombinant PRL phosphatases in vitro. A. relative activities of GST fusion proteins of PRLs in dephosphorylating a phosphotyrosine peptide in the presence of PE. B shows relative activities of GST/PRL-3 fusion protein pre-incubated with PE and then washed (+) or not washed (−) were determined using the peptide substrate. C shows relative activities of GST-PRL-3 in dephosphorylating DiFMUP in the presence of PE. Data represent mean±s.d. values of triplicate samples.

FIG. 3 illustrates that pentamidine inactivates intracellular PRLs in NIH3T3 transfectants. A shows PTPase activities of α-Flag immunocomplexes from untreated (0) or PE-treated (5 min) NIH3T3 transfectants of the control vector (V) or Flag-PRL-1 expression construct. B shows relative amounts of Flag-PRL-1 in the immunocomplexes in (A) as detected by Western blotting. C shows PTPase activities of α-Flag immunocomplexes from untreated or PE-treated NIH3T3 transfectants of Flag-PRL-2. D shows relative amounts of Flag-PRL-2 in the immunocomplexes as determined by Western blotting. E shows PTPase activities of α-Flag immunocomplexes from untreated or PE-treated NIH3T3 transfectants of Flag-PRL-3. F shows relative amounts of Flag-PRL-3 in the immunocomplexes as determined by Western blotting. Data of PTPase activity represent mean±S.D. of triplicate samples.

FIG. 4 illustrates the duration of pentamidine-induced inactivation of PRL-2 in NIH3T3 transfectants. A shows relative PTPase activity of anti-Flag immunocomplexes from NIH3T3 transfectant of Flag-PRL-2 untreated or treated with PE for 5 minutes, washed to remove cell-free drug and then incubated for various times prior to cell lysis. Data represent mean±s.d. values of triplicate samples. B shows relative amounts of Flag-PRL-2 in the immunocomplexes as determined by SDS-PAGE/Western blotting.

FIG. 5 illustrates that pentamidine inhibits the growth of WM9 human melanoma tumors in nude mice. A Tumor volumes in nude mice inoculated with WM9 cells (s.c.) at the flanks and subjected to no treatment (Control) or treatment with PE (0.25 mg/mouse, i.m., every two days in the hip area) were measured on the dates as indicated. Data represent mean±SEM (n=8).

FIG. 6 illustrates that pentamidine treatment induces necrosis of WM9 tumors in nude mice. A and B show representative views (×4) of hematoxylin and eosin stained sections of WM9 tumors in nude mice without treatment at the 4^{th} week following inoculation (A) and WM9 tumors in nude mice treated with PE for 16 weeks (B). C is a higher power view (×40) of the tumor illustrated in B.

FIG. 7 illustrates that pentamidine induces cellular protein tyrosine phosphorylation and inhibits PRL-2 phosphatase in WM9 cells. A shows relative PTPase activities of α-Flag immunocomplexes from WM9 transfectants of the control vector (V) or Flag-PRL-2 expression construct treated with PE for 5 minutes. B shows relative amounts of Flag-PRL-2 in the immunocomplexes as determined by Western blotting. C. relative PTPase activities of α-SHP-2 immunocomplexes from WM9 cells treated with PE for 5 minutes. D shows relative amounts of SHP-2 in the immunocomplexes as determined by Western blotting. E Total cell lysates of WM9 cells treated with PE (5 min) were analyzed by Western blotting with antibodies as indicated. The positions of protein markers (kDa) are indicated on the left.

FIG. 8 illustrates that pentamidine inhibits the in vitro growth of human cancer cell lines that express PRLs. A-F Growth of cell lines of different human malignancies cultured in the absence or presence of various amounts of PE for 6 days was determined by standard MTT assays. Data represent mean±s.d. values of triplicate samples. G shows expression of transcripts of PRLs in the cell lines and in peripheral blood mononuclear cells (PBMC) from two healthy volunteers as determined by RT-PCR.

FIG. 9 illustrates that pentamidine inhibits the in vitro growth of human cancer cell lines that express PRLs. Growth of cell lines of different human malignancies cultured in the absence or presence of various amounts of PE for 6 days was determined by MTT assays. Cell lines were Burkitts lymphoma (A), multiple myeloma (IM9 cells) (B), colon adenocarcinoma (LOVO cells) (C), neuroblastoma (SK—N—SH cells) (D), T-ALL (PEER cells) (E), glioma (U251 cells) (F), multiple myeloma (U266 cells) (G) and T-lymphoma (H9 cells) (H). Data represent mean—s.d. values of triplicate samples.

FIG. 10 illustrates the amino acid sequence of human wild type PRL-1 (A) (SEQ ID NO: 1) and mutant PRL-1R86 (B) (SEQ ID NO: 4); the human wild type PRL-2 (C) (SEQ ID NO: 2) and mutant PRL-2R83 (D) (SEQ ID NO: 5); and the human wild type PRL-3 (E) (SEQ ID NO: 3) and mutant PRL-3R86 (F) (SEQ ID NO: 6). The mutants were generated through introducing a single nucleotide change in the cDNA of the wild types via recombinant DNA technology. The conserved regions between the wild type and mutant pairs are underlined.

FIG. 11 illustrates the identification of the human PRL-1S86 (serine) counterpart residue in human PRL-2 and human PRL-3 based on conserved flanking residues and generation of the PRL-1R86, PRL-2R83 and PRL-386 mutants. A shows conserved residues in wild-type PRLs. B shows conserved residues in mutant PRLs.

FIG. 12 illustrates that PRL-1R86 mutant has PTPase activity comparable to that of PRL-1 and is insensitive to pentamidine inhibition. A PRL-1R86 is a PRL-1 mutant, which contains a single amino acid residue substitution (a serine to arginine) at position 86 in the PTPase domain. B shows PTPase activities of GST (control) or GST fusion proteins (10 ng/reaction) of PRL-1 or PRL-1R86 (R86) in the absence or presence of PE as determined by PTPase assays using a phosphotyrosine peptide substrate. Data represent mean±s.d. values of triplicate samples. C shows relative amounts of Flag-tagged PRL-1 (Flag-PRL-1) or PRL-1R86 (Flag-R86) in immunocomplexes from WM9 transfectants untreated (0) or treated with PE (5 min) as determined by Western blotting. D shows PTPase activities of the immunocomplexes as determined by PTPase assays using the peptide substrate. Data represent mean±s.d. values of triplicate samples.

FIG. 13 illustrates that a PE-insensitive PRL-1R86 mutant (1R) confers resistance to PE-induced growth inhibition in WM9 melanoma cells. Stable WM9 transfectants of expression constructs of employing Flag-tagged PRL-1 or PRL-1R86 were generated. For measurement of pentamidine effects on cell growth in vitro, cells were cultured in the absence (−) or presence (+) of various amounts of pentamidine for 6 days with viable cells quantified by MTT assays as described. A shows the results of recombinant protein PTPase assays. B shows the results of immunocomplex PTPase assays. C. immunocomplex Western blotting. D. Cell growth assays (MTT). Data represent mean±s.d. of triplicate samples.

FIG. 14 illustrates a PE-induced growth inhibition of WM9 transfectants of control vector (V), Flag-PRL-1 or Flag-PRL-1R86 in day 6 culture that was determined by MTT assays (A). B. The transfectants of A showed similar growth rates in day 6 culture in the absence of PE. C. A PE-induced growth inhibition of DU145 and DU145R cells in day 6 cultures as determined by MTT assays. Data represent mean±s.d. values of triplicate samples.

FIG. 15 illustrates that PE lacks inhibitory activity against recombinant PTPalpha and cdc25 in vitro. A. Relative PTPase activities of GST fusion proteins of PRL-2 or PTPalpha (10 ng/reaction) in the absence or presence of PE. B. Relative PTPase activities of GST fusion proteins of PRL-2 or cdc25 (10 ng/reaction) in the absence or presence of PE. Data represent mean±s.d. values of triplicate samples.

FIG. 16 illustrates that PE selectively quenches the intrinsic fluorescence of recombinant PRL-1 but not PRL-1R86 mutant. A. PRL-1 and PRL-1R86 proteins were separated in SDS-PAGE and detected by Coomassie blue staining. B. PTPase activities of the proteins in the absence or presence of PE. C. Fluorescence of PRL-1 in the absence or presence of PE as determined by fluorescence photospectrometry. D. Fluorescence of PRL-1R86 in the absence or presence of PE. Data (B-D) are mean±s.d. values of triplicate samples.

FIG. 17 compares the activities of PE and PR (propamidine). A. Chemical structures of PE and PR. B. Relative PTPase activities of his-PRL-1 in the absence or presence of PE or PR. C. Intrinsic fluorescence of his-PRL-1 in the absence or presence of PR as determined by fluorescent photospectrometry. Data represent mean±s.d. of triplicate samples.

FIG. 18 illustrates in vitro growth inhibition of WM9 cells cultured in the presence of PE, IFNα or both for 6 days as determined by MTT assays (A). Data represent mean±s.d. values of triplicate samples. B. IFNα-induced Stat1 tyrosine phosphorylation in WM9 cells cultured in the absence or presence of PE as determined by SDS-PAGE/Western blotting using antibodies as indicated.

FIG. 19 illustrates the structures of PRL-2 and PRL-2R83 (A). B. PTPase activities of GST (control) or GST fusion proteins (10 ng/reaction) of PRL-2 or PRL-2R83 as determined by PTPase assays using a phosphotyrosine peptide substrate. C. Relative PTPase activities of PRL-2 and PRL-2R83 in the absence or presence of PE. D. Structures of PRL-3 and PRL-3R86. E. PTPase activities of GST (control) or GST fusion proteins (10 ng/reaction) of PRL-3 or PRL-3R86 as determined by using the peptide substrate. F. Relative PTPase activities of PRL-3 and PRL-3R86 in the absence or presence of PE. Data represent mean±s.d. values of triplicate samples.

### DETAILED DESCRIPTION OF THE INVENTION

Embodiments of the present invention provide therapeutic compositions and methods useful in the prevention, treatment or amelioration of cancer. By “cancer” is meant any malignant neoplasm, defined as an abnormal mass of tissue, the growth of which exceeds and is uncoordinated with that of the normal tissues and persists in the same excessive manner after cessation of the stimulus which evoked the change. By “prophylactic” or “prevention,” it is meant the protection, in whole or in part, against a particular disease or a plurality of diseases. By “therapeutic,” it is meant the amelioration of the disease itself, and the protection, in whole or in part, against further disease. By “amelioration” is meant improvement in the course of the disease including, but not limited to, alleviation of symptoms, improvement of the patient's condition, and the like.

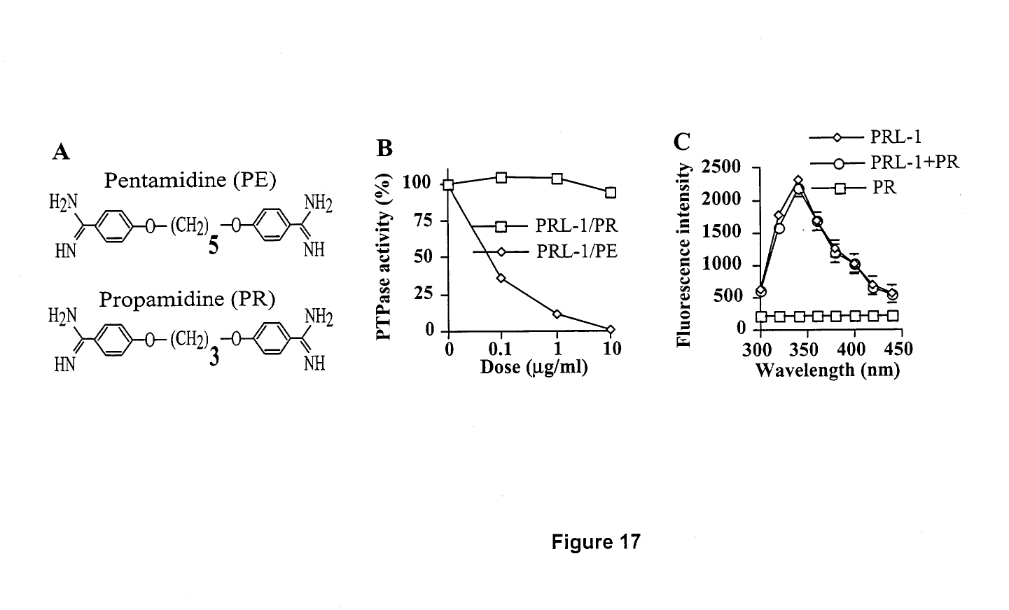

Pentamidine or “PE,” as used herein, is 1,5-di(4-amidinophenoxy)pentane, and is meant to encompass a pharmaceutically acceptable analogue or prodrug thereof, or a pharmaceutically acceptable salt thereof, and biological equivalents which are effective in inhibiting protein tyrosine phosphatases. It shall be understood that the prodrug used must be one that can be converted to an active agent in or around the site to be treated. The structure of pentamidine, an aromatic diamidine, is illustrated in FIG. 17A.

Pentamidine has been in clinical use for more than 60 years as an anti-protozoan drug although its mechanism of action remains elusive. It is used for the treatment of leishmaniasis, the hemolymphatic stage of Gambian trypanosomiasis, and Pneumocystis carinii pneumonia (PCP). Several putative mechanisms of action of the drug had been proposed but were unsubstantiated or irrelevant to its efficacy. For example, a reported inhibitory activity of PE against constitutive brain nitric oxide synthase was only effective at a 100-1000 μM range, much higher than a therapeutically achievable level. The therapeutic dosage of the drug in humans as an anti-protozoan is 2-4 mg/kg. The drug is known to have DNA binding activity that was found to be unrelated to its anti-PCP action or pharmacological efficacy. Several lines of evidence suggest that PE action against leishmaniasis, a tropic disease caused by proliferation of leishmania pathogen in host macrophages, might be mediated via targeting molecules in host cells and involve host immune system. For example, PE selectively kills intracellular but not the free-living form of leishmania. The drug was also found to have little anti-leishmania activity in T cell-deficient mice. In contrast, the anti-leishmania drug amphotericin B acts against both forms of the protozoa and is active in normal as well as immune deficient mice. These observations indicate an indirect action mechanism of PE that acts against host cellular targets and depends on host immunity, distinctive from the direct action of amphotericin B against the pathogen. In another study, we demonstrated that another anti-leishmania drug, sodium stibogluconate (SSG or SS), showed similar characteristics to PE in its anti-leishmania action, although it is an organic antimony compound chemically unrelated to PE.

We have unexpectedly discovered that pentamidine, at therapeutic dosages, is a potent inhibitor of selective PTPases, including oncogenic PRL phosphatases through direct binding to the PTPases, and has therapeutic potential against malignancies associated with PRL over-expression. Further, it was unexpectedly discovered that the PE shows in vitro growth inhibitory activity against human cancer cell lines expressing PRLs, and in in vivo mouse models against human cancer cell lines expressing PRLs. Without being bound by theory, it is believed that the growth inhibitory activity of PE against human cancer cells is likely to be mediated at least in part via inactivating PRL-1. We discovered that inhibition of PRLs by pentamidine resulted from direct interaction between PE and the target PTPase depending on a specific chemical feature in PE and a unique residue in the phosphatase.

Pentamidine at 1-10 μg/ml effectively inhibited recombinant PRLs in dephosphorylating a phosphotyrosine peptide substrate in vitro. Moreover, intracellular PRLs from NIH3T3 transfectants briefly treated with pentamidine (1 or 10 μg/ml) were inactivated and required more than 24 hours for their full recovery. The drug also inactivated PRL-2 in WM9 melanoma cells, demonstrating its effectiveness in targeting the oncogenic phosphatase in human malignant cells. Importantly, the inhibitory activity of the drug was shown to be restricted to a subset of PTPases in cancer cells as pentamidine treatment under comparable conditions failed to inactivate SHP-2 PTPase in WM9 cells. The fact that recombinant SHP-2 was also insensitive to the drug in vitro suggests a correlation of in vitro and in vivo sensitivities of PTPases to the drug. Thus the in vitro sensitive PTP1B might also be a target of pentamidine in vivo.

Pentamidine at the dosage of approximately 10 mg/kg inhibited the growth of WM9 melanoma tumors in nude mice and kept the tumors volumes at levels similar to those at the treatment initiation point during the 16 week study period. This is striking in comparison to the aggressive growth of WM9 tumors in the untreated mice that resulted in the termination of the animals 4 weeks after tumor inoculation. The dosage used in this study is similar to the therapeutic dose of the drug in humans (about 1 to about 10 mg/kg, and more preferably about 2 to about 4 mg/kg) and did not result in histologic abnormalities in the animals. Given that pentamidine inhibited the growth of cell lines of other human malignancies as it did against WM9 cells in culture, the drug acts against tumors of these additional cancer cell lines in vivo and therefore has activity against different types of cancers. These results suggest the potential of this drug, already in use clinically as an anti-protozoan, for rapid incorporation into current anti-cancer therapies.

Data from our present studies provide evidence that the anti-cancer activity of pentamidine is mediated via inactivation of cancer cell-expressed PTPases, in particular the oncogenic PRLs, and resulted in preferential killing of the malignant cells. PRLs in NIH3T3 fibroblasts and PRL-2 in WM9 melanoma cells were inactivated by pentamidine at 1 μg/ml. Such a dose was likely within the in vivo drug levels in the nude mice treated with pentamidine (˜10 mg/kg) based on its tissue disposition at 1.6-34 μg/g of tissue in the major organs of rats 24 hours following pentamidine injection (4 mg/kg). Thus inactivation of PRLs occurs in WM9 tumor cells in the pentamidine-treated mice. Given the detected expression of PRLs in WM9 cells and the other cancer cell lines, and the known oncogenic potential of the phosphatases, PRLs could be among the key targets of the drug in mediating its anti-cancer activity.

Moreover, the significant tumor cell necrosis in mice treated with pentamidine at a nontoxic dose indicates that the drug selectively caused the death of the malignant cells with no serious effects on the normal cells in the animals. Such a putative mode of action of the drug predicts that human malignancies associated with over-expression of the PRL phosphatases are sensitive to pentamidine therapy and indicates the value of PRLs as markers for identification of pentamidine-sensitive tumors.

Additionally, cancers unresponsive to conventional therapies might be sensitive to pentamidine as an alternative treatment given that the drug targets molecules different from those of the conventional therapies. In this regard, the duration of pentamidine-induced PRL-2 inactivation as defined in our studies could be important as it provides a basis for rational design of PRL-targeted pentamidine therapy in cancer treatment.

Our finding that pentamidine irreversibly inhibits recombinant PRLs and has lasting effects on intracellular PRL-2 provides insights into the inhibitory mechanism of the drug against PTPases. It reveals that inactivation of PTPases by pentamidine involves a tight binding of the inhibitor to the enzymes and/or covalent modification of the phosphatases by the drug. Such models are consistent with an increase of molecular mass of recombinant PRL-2 incubated with pentamidine in vitro that was detected by mass spectrometry. Furthermore, the potent effects of pentamidine in inactivating PRLs not only designate the drug as the first clinically usable PRL inhibitor but also indicate its potential value as an experimental tool in elucidating the physiologic function and oncogenic mechanism of PRLs that have not been defined so far.

The observation that pentamidine at nontoxic doses is a potent inhibitor of PRLs and PTP1B is also significant in that it opens up several exciting avenues in developing PTPase-targeted therapeutics with low toxicity. Since pentamidine, as shown in FIG. 17, is a chemically defined compound with a number of its derivatives already reported (See Tidwell, R. R., Jones, S. K. Geratz, J. D., Ohemeng, K. A., Bell, C. A., Berger, B. J. and Hall, J. E. Development of pentamidine analogues as new agents for the treatment of Pneumocystis carinii pneumonia, Ann. N.Y. Acad. Sci., 616: 421-41, 1990 and Donker, I. O. and Clark, A. M., In vitro antimicrobial activity of aromatic diamidines and diimidazolines related to pentamidine, Eur. J. Med. Chem., 34: 639-43, 1999, the disclosures of which are hereby incorporated by reference) or could be easily synthesized, screening such derivatives might lead to more specific and effective inhibitors of individual PRLs. It also allows structural analysis of PRLs in complex with pentamidine that could provide a basis for rational design of next generations of inhibitors against these oncogenic phosphatases. Given the observed involvement of PRLs in human malignancies, it could be expected that mono-specific inhibitors of the phosphatases will have significant value as anti-cancer therapeutics. Similar approaches could also be applied to develop specific inhibitors of PTP1B or other PTPases as targeted novel therapeutics.

The active pentamidine compositions described herein include all biochemical equivalents thereof (i.e., salts, precursors, the basic form, and the like), including derivatives thereof such as, but not limited to, derivatives described by Tidwell, R. R. et al (supra) and Donker, I. O. et al. (supra). The effective amount of pentamidine to be administered to a mammal, especially a human, can be any non-toxic, clinically-tolerated dosage. The term “clinically-tolerated” is meant to have its normal meaning as understood by medical practitioners. A suitable non-toxic dosage of pentamidine as an anti-protozoon in humans is about 2 to about 4 mg/kg. However, this dosage is not intended to be limiting to the invention, as any non-toxic, clinically-tolerated dosage may be considered more appropriate by a practitioner using routine knowledge and experimentation.

The compositions can be administered by any suitable route. The manner in which the agent is administered is dependent, in part, upon whether the treatment is prophylactic or therapeutic. Although more than one route can be used to administer a particular therapeutic composition, a particular route can provide a more immediate and more effective reaction than another route. Accordingly, the described routes of administration are merely exemplary and are in no way limiting.

The composition(s) is preferably administered as soon as possible after it has been determined that an animal, such as a mammal, specifically a human, is at risk for cancer. Treatment will depend, in part, upon the particular therapeutic composition used, the amount of the therapeutic composition administered, the route of administration, and the cause and extent, if any, of the disease.

The dose administered to an animal, particularly a human, should be sufficient to effect the desired response in the animal over a reasonable period of time. The dosage will depend upon a variety of factors, including the strength of the particular therapeutic composition employed, the age, species, condition or disease state, and body weight of the animal. The size of the dose also will be determined by the route, timing and frequency of administration as well as the existence, nature, and extent of any adverse side effects that might accompany the administration of a particular therapeutic composition and the desired physiological effect. Various conditions or disease states, in particular, chronic conditions or disease states, may require prolonged treatment involving multiple administrations.

Suitable doses and dosage regimens can be determined by conventional range-finding techniques. Generally, treatment is initiated with smaller dosages, which are less than the optimum dose of the compound. Thereafter, the dosage is increased by small increments until the optimum effect under the circumstances is reached.

The administration(s) may take place by any suitable technique, including oral, subcutaneous and parenteral administration. Non-limiting examples of parenteral administration include intravenous, intra-arterial, intramuscular and intraperitoneal routes. The dose and dosage regimen will depend mainly on whether the inhibitors are being administered for therapeutic or prophylactic purposes, separately or as a mixture, the type of biological damage and host, the history of the host, and the type of inhibitors or biologically active agent. The amount must be effective to achieve an enhanced therapeutic index. It is noted that humans are generally treated longer than the mice and rats with a length proportional to the length of the disease process and drug effectiveness. The doses may be single doses or multiple doses over a period of several days. Therapeutic purposes are achieved as defined herein when the treated hosts exhibit improvement against disease or infection, including but not limited to improved survival rate, more rapid recovery, or improvement or elimination of symptoms. If multiple doses are employed, as preferred, the frequency of administration will depend, for example, on the type of host and type of cancer, dosage amounts, and the like.

Compositions for use in the present inventive method preferably comprise a pharmaceutically acceptable carrier and an amount of the therapeutic composition sufficient to treat the particular disease prophylactically or therapeutically. The carrier can be any of those conventionally used and is limited only by chemical-physical considerations, such as solubility and lack of reactivity with the compound, and by the route of administration. It will be appreciated by one of ordinary skill in the art that, in addition to the following described pharmaceutical compositions, the therapeutic composition can be formulated as polymeric compositions, inclusion complexes, such as cyclodextrin inclusion complexes, liposomes, microspheres, microcapsules and the like.

The therapeutic composition can be formulated as a pharmaceutically acceptable acid addition salt. Examples of pharmaceutically acceptable acid addition salts for use in the pharmaceutical composition include those derived from mineral acids such as, but not limited to, hydrochloric, hydrobromic, phosphoric, metaphosphoric, nitric and sulfuric acids, and the like, and organic acids such as, but not limited to, tartaric, acetic, citric, malic, lactic, fumaric, benzoic, glycolic, gluconic, succinic, and arylsulphonic, for example p-toluenesulphonic, acids, and the like.

The pharmaceutically acceptable excipients described herein, for example, vehicles, adjuvants, carriers or diluents, are well-known to those who are skilled in the art and are readily available to the public. It is preferred that the pharmaceutically acceptable carrier be one which is chemically inert to the therapeutic composition and one which has no detrimental side effects or toxicity under the conditions of use.

The choice of excipient will be determined in part by the particular therapeutic composition, as well as by the particular method used to administer the composition. Accordingly, there are a wide variety of suitable formulations of the pharmaceutical composition of the present invention. The formulations described herein are merely exemplary and are in no way limiting.

Injectable formulations are among those that are preferred in accordance with the present inventive method. The requirements for effective pharmaceutically carriers for injectable compositions are well-known to those of ordinary skill in the art (see Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986)). It is preferred that such injectable compositions be administered intramuscularly, intravenously, or intraperitoneally.

Topical formulations are well-known to those of skill in the art. Such formulations are suitable in the context of the present invention for application to the skin in a form such as, but not limited to, patches, solutions, ointments, and the like.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The inhibitor can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride, with or without the addition of a pharmaceutically acceptable surfactant, such as a soap or a detergent, suspending agent, such as pectin, carbomers, methylcellulose, hydroxypropylmethyl-cellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metals, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-p-aminopropionates, and 2-alkyl-imidazoline quaternary ammonium salts, and (e) mixtures thereof. The parenteral formulations will typically contain from about 0.5 to about 25% by weight of the active ingredient in solution. Preservatives and buffers may be used.

In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The present inventive method also can involve the co-administration of other pharmaceutically active compounds. By “co-administration” is meant administration before, concurrently with, e.g., in combination with anti-cancer composition in the same formulation or in separate formulations, or after administration of a therapeutic composition as described above. For example, corticosteroids, e.g., prednisone, methylprednisolone, dexamethasone, or triamcinalone acetinide, or noncorticosteroid anti-inflammatory compounds, such as ibuprofen or flubiproben, can be co-administered. Similarly, vitamins and minerals, e.g., zinc, anti-oxidants, e.g., carotenoids (such as a xanthophyll carotenoid like zeaxanthin or lutein), and micronutrients can be co-administered. In addition, other types of inhibitors of the protein tyrosine phosphatase pathway.

### EXAMPLES

The examples described herein are intended to be illustrative, but not limiting, as one skilled in the art would recognize from the teachings hereinabove and the following examples, that other derivatives of PE, other dosages of PE, other cancer cell lines, substrates, reagents, methods, and the like, without limitation, may be employed, without departing from the scope of the invention as claimed.

The following reagents, assays, cells and cell lines, and animals were employed in the examples below.

Reagents

Pentamidine (pentamidine isethionate, Pentam 300) was purchased from American Pharmaceutical Partners, Inc. (Schaumberg, Ill.).

SSG and GST fusion proteins of SHP-1 , SHP-2, PTP1B and MKP1 have been described previously in copending applications incorporated by reference herein. SHP-2 is used herein to mean Src-homology protein tyrosine phosphatase-2 domain; SHP-1 is used herein to mean Src-homology protein tyrosine phosphatase 1 domain; and SSG is used herein to mean sodium stibogluconate.

DNAs of human PRL-1, PRL-2 and PRL-3 coding region were derived by RT-PCR from H9 cells and inserted in frame into the pGEX vector. GST fusion proteins of the PRL phosphatases were prepared from DH5α bacteria transformed with the pGEX fusion protein constructs following established procedures.

cDNAs encoding the PRLs tagged at the N-termini with the Flag epitope were generated via recombinant DNA technique, sequenced to confirm their identities and cloned into the pBabepuro or pRK5 vector.

Anti-Flag monoclonal antibody (M2, Sigma), anti-phosphotyrosine monoclonal antibody (4G10, Upstate Group Inc., Charlottesville, Va.), anti-β-actin monoclonal antibody (Pharmacia) and anti-SHP-2 polyclonal antibodies (Santa Cruz Biotechnology Inc., Santa Cruz, Calif.) were purchased from the commercial sources.

A synthetic phosphotyrosine peptide (R-R-L-I-E-D-A-E-pY-A-A-R-G, Upstate Group Inc.) (SEQ ID NO: 7) and DiFMUP (6,8-difluoro-4-methylumbelliferyl phosphate, (Molecular Probes, Eugene, Oreg.) were purchased as substrates for PTPase assays.

Assays

In vitro PTPase assays and immunocomplex PTPase assays. In vitro PTPase assays were used to determine the effects of compounds on recombinant PTPases, following established procedures using a synthetic phosphotyrosine peptide or DiFMUP as the substrate. The assays were conducted in the absence (−) or presence (+) of inhibitory compounds with the relative PTPase activities calculated (±×100%). To assess the reversibility of PTPase inhibition, GST fusion proteins of the PTPases bound on glutathione beads (Pharmacia) were pre-incubated with cold Tris buffer (50 mM Tris, pH 7.0) or Tris buffer containing the inhibitor at 4° C. for 30 minutes. The beads were then washed three times in cold Tris buffer or not washed prior subjecting to in vitro PTPase assays.

Immunocomplex PTPase assays were performed to assess the effects of pentamidine on intracellular PTPases. Individual PTPases were immunoprecipitated from untreated or pentamidine-treated cells that were washed with fresh medium and then lysed in cold lysis buffer (50 mM Tris, pH 7.4; 150 mM NaCl; 1% NP40; 2 mM PMSF; 20 μg/ml of Aprotinin). The immunocomplexes were collected with protein G. sepharose beads (Pharmacia) and washed in cold lysis buffer for 4 times. Approximately 90% contents of individual samples were split into 3 comparable portions and each was then incubated in 50 μl of PTPase buffer (50 mM Tris, pH 7.4; 0.2 mM phosphotyrosine peptide) at 22° C. for 18 hours. 100 μl of malachite green solution (Upstate Group, Inc.) was added to each reaction, which was then incubated at 22° C. for 5 minutes prior to measurement of OD660 to quantify the amounts of free phosphate cleaved by the PTPases from the peptide substrate. The remaining 10% contents of individual samples were analyzed by SDS-PAGE/Western blotting to quantify the relative amounts of the phosphatase proteins. To assess the duration of pentamidine effects on the activities of intracellular PTPases, Flag-PRL-2 transfected cells were untreated or treated with pentamidine (1 mg/ml) for 5 minutes at 37° C., washed twice with culture medium to remove cell-free drug and then incubated in fresh culture medium at 37° C. for 24-72 hours prior to termination by lysing the cells in cold lysis buffer. Flag-PRL-2 were immunoprecipitated from the lysates and subjected to PTPase assays and SDS-PAGE/Western blotting as described above.

Cells, cell culture, cell growth assays and transfection. NIH3T3, WM9, DU145, C4-2, Hey, SW480, A549 cell lines are described in the copending applications incorporated by reference herein. The cell lines were cultured in RPMI 1640 supplemented with 10% fetal calf serum (FCS). For measurement of pentamidine effects on cell growth in vitro, cells were cultured in the absence (−) or presence (+) of various amounts of pentamidine for 6 days with viable cells quantified by MTT assays as described. Percentages of growth inhibition by pentamidine were calculated (±×%)

The effects of pentamidine on intracellular PTPases were assessed using NIH3T3 or WM9 transfectants. NIH3T3 cells were transfected with the pBabepuro vector (V) or pBabepuro expression constructs of Flag-tagged PRLs using Lipofectamine (BRL) following the manufacturer's procedures. Transfectants were selected in the presence of puromycine (0.5 μg/ml) for two weeks and expanded in culture without puromycine prior to their usage in measuring the effects of pentamidine on the PTPase activities of intracellular Flag-PRLs. WM9 cells were transfected with the pRK5 vector or pRK5 expression construct of Flag-tagged PRL-2 using Lipofectamine. The cells were used at 48 hours post-transfection for measuring the effects of pentamidine on the PTPase activities of intracellular Flag-PRL-2.

Animal Studies. Athymic nude mice of 4 weeks old (Taconic Farms Inc.) were subcutaneously inoculated in the flanks with WM9 human melanoma cells (4×10⁶ cells/site) on day 0. Starting on day 2, the mice were subjected to no treatment (control) or treatment with pentamidine (0.25 mg/mouse, every two days) intramuscularly injected at the hip area. Tumor volume was calculated using the formula for a prolate spheroid (V =4/3 πa ²b) and presents as mean±SEM (n=8). Mouse viability and body weights were recorded weekly. H.E. (hematoxylin and eosin) stained tissue sections of internal organs and tumor inoculation sites tissues of the mice were prepared and subjected microscopic evaluation.

Detection of induced cellular protein tyrosine phosphorylation. WM9 cells were untreated or treated with various amounts of pentamidine for 5 minutes at 37° C. Cells were lysed in cold lysis buffer (50 mM Tris, pH 7.4; 150 mM NaCl; 0.2 mM Na₃VO₄; 20 mM NaF; 1% NP40; 2 mM PMSF; 20 μg/ml of Aprotinin and 1 mM of sodium molybdic acid). Cell lysates were separated in 10% SDS-PAGE gels, transferred to nitrocellulose membrane (Schleicher & Schuell), probed with specific antibodies and detected using an enhanced chemiluminescence kit (ECL, Amersham).

RT-PCR analysis of the expression levels of PRL phosphatases. Expression of the transcripts of PRLs in peripheral blood mononuclear cells (PBMC) from two healthy volunteers and in cancer cells lines were detected by RT-PCR with specific primer pairs for individual PRLs as listed below or for GAPDH. RT-PCR products were separated in an agarose gel and visualized by ethidium bromide staining with their identities confirmed by restriction endonuclease mapping. The sequence of primer pairs are:

| | | |
|---|---|---|
| huPRL-3/5, | 5′-TAGGATCCCGGGAGGCGCCATGGCTCGGATGA-3′; | (SEQ ID NO: 8) |
| huPRL-3/3, | 5′-GAGTCGACCATAACGCAGCACCGGGTCTTGTG-3′; | (SEQ ID NO: 9) |
| huPRL-2/5, | 5′-TAGGATCCCCATAATGAACCGTCCAGCCCCTGT-3′; | (SEQ ID NO: 10) |
| huPRL-2/3, | 5′-GAGTCGACCTGAACACAGCAATGCCCATTGGT-3′; | (SEQ ID NO: 11) |
| huPRL-1/5, | 5′-TAGGATCCCCAACATGGCTCGAATGAACCGCCC-3′; | (SEQ ID NO: 12) |
| huPRL-1/3, | 5′-GAGTCGACTTGAATGCAACAGTTGTTTCTATG-3′. | (SEQ ID NO: 13) |

### Example 1

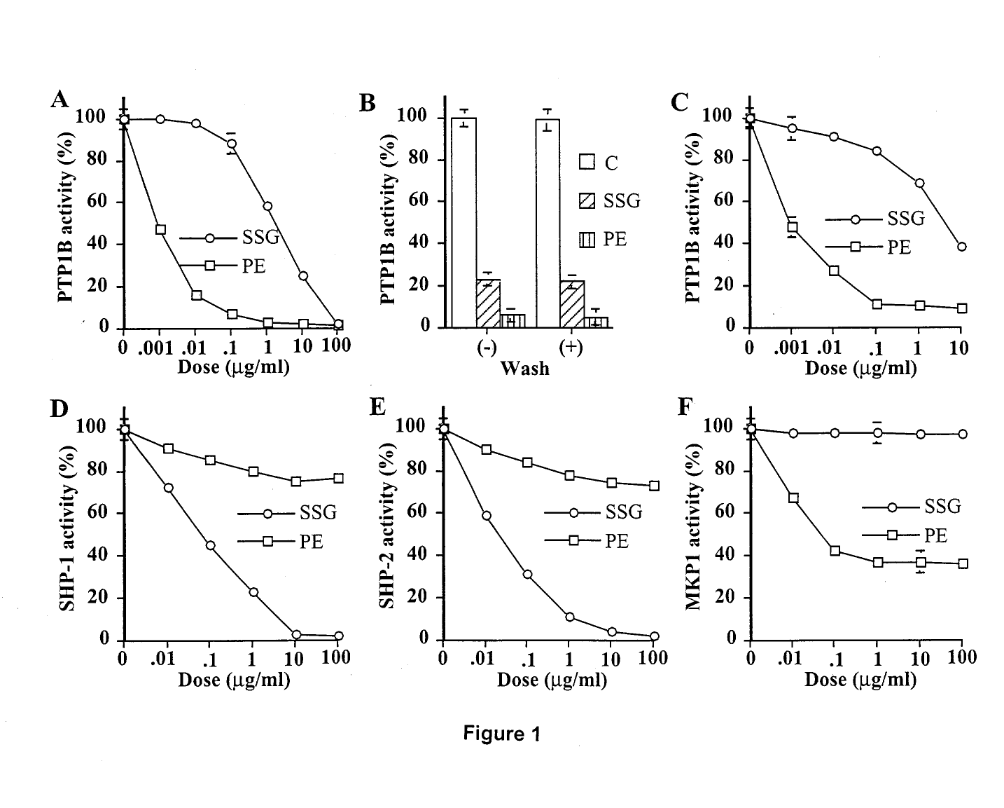

Pentamidine has differential inhibitory activities against PTPases in vitro. Activities of GST fusion proteins of PTP1B (FIG. 1A), SHP-1 (FIG. 1D), SHP-2 (FIG. 1E), MKP1 (FIG. 1F) in dephosphorylating a phosphotyrosine peptide in the absence or presence of various amounts of pentamidine or SSG were measured in in vitro PTPase assays. As illustrated in FIG. 1A, pentamidine inhibited recombinant PTP1B in dephosphorylating a phosphotyrosine peptide in vitro in a dose-dependent manner and achieved near complete inactivation of PTP1B at 1 μg/ml while a similar degree of inhibition required 100 fold amount of the known PTPase inhibitor SSG. In FIG. 1B, relative activities of GST/PTP1B fusion protein pre-incubated with pentamidine (1μg/ml) or SSG (10 μg/ml) and then washed (+) or not washed (−) were determined using the peptide substrate. This inhibition of PTP1B by pentamidine in vitro was irreversible as it was not abolished by a washing process effective against the reversible inhibitor suramin. As shown in FIG. 1C, in vitro activities of GST/PTP1B fusion protein in dephosphorylating DiFMUP, an alternative substrate, in the absence or presence of pentamidine or SSG were also determined. Pentamidine inhibited the activity of GST/PTP1B to a much larger extent than SSG. FIGS. 1D and 1E, respectively, show that pentamidine has little activity against recombinant SHP-1 and SHP-2, which were sensitive to SSG. In FIG. 1F, the activity of recombinant MKP1 was partially inhibited by pentamidine but not by SSG. Data represent mean±s.d. values of triplicate samples.

These results demonstrate that pentamidine is an inhibitor of selective PTPases in vitro with a specificity profile different from that of SSG.

### Example 2

Pentamidine is a potent inhibitor of recombinant PRL phosphatases in vitro. Given the potential pathogenic role of over-expression of PRL phosphatases in human malignancies, these oncogenic phosphatases are highly attractive targets for developing inhibitors as novel anti-cancer therapeutics. Clinically usable inhibitors of the PRLs have not been reported.

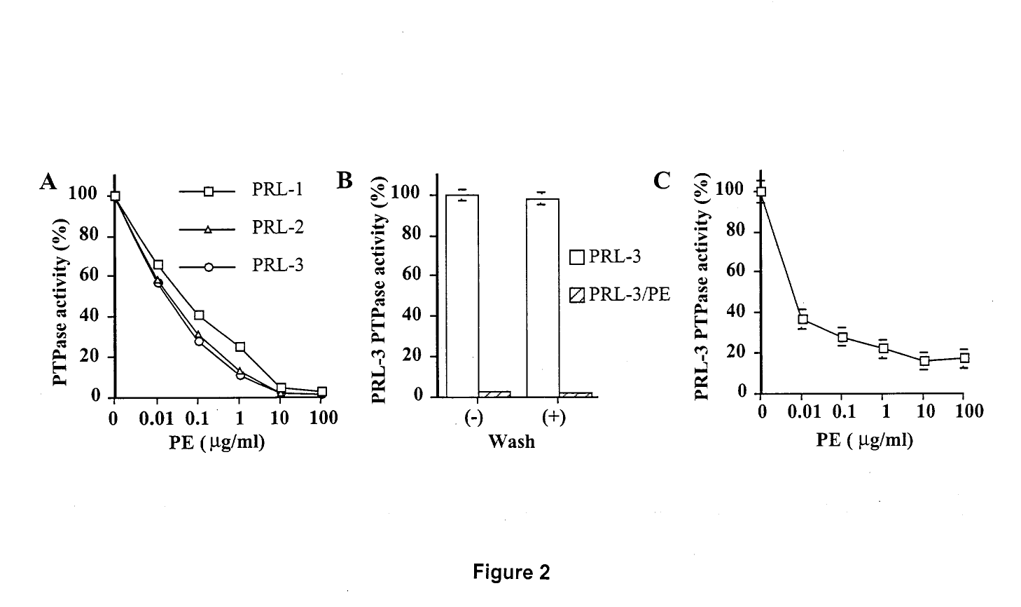

In FIG. 2A, the activities of recombinant PRL-1, PRL-2 and PRL-3 in dephosphorylating a phosphotyrosine peptide substrate in vitro were almost equally inhibited by pentamidine in a dose-dependent manner with nearly complete inactivation of the phosphatases at 10 μg/ml. In FIG. 2B, washing PRL-3 pre-incubated with pentamidine failed to remove the inhibition, indicating an irreversible action of the drug. As illustrated in FIG. 2C, the inhibitory activity of pentamidine against recombinant PRL-3 was confirmed using DiFMUP, as an alternative substrate, in in vitro PTPase assays. Data represent mean±s.d. values of triplicate samples.

### Example 3

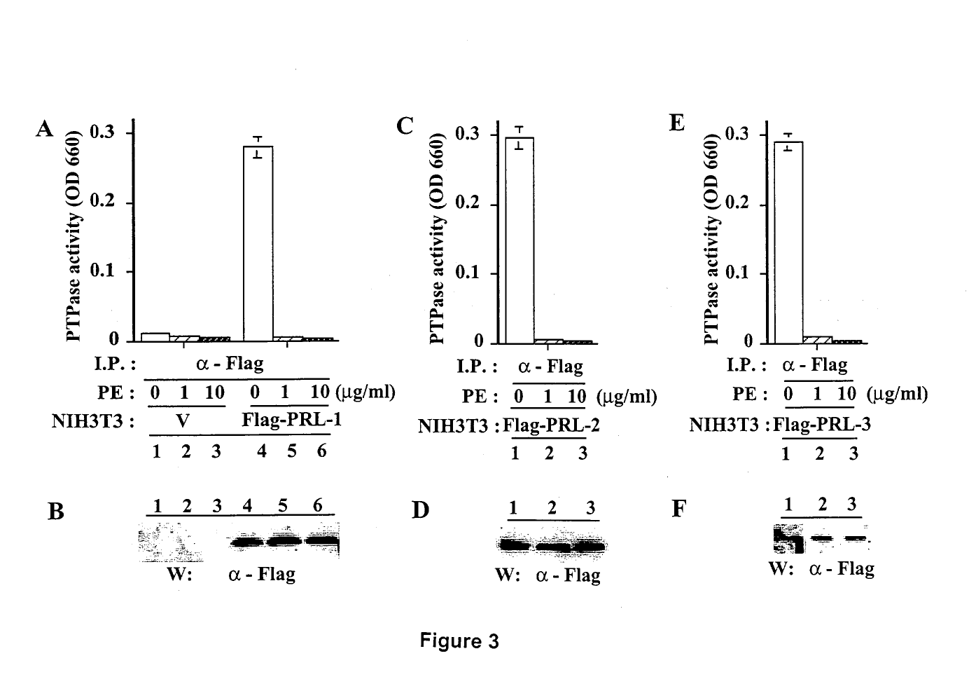

Pentamidine at therapeutic dosage is an effective inhibitor of intracellular PRLs in NIH3T3 cells. The effects of pentamidine against intracellular PRL phosphatases were assessed. To circumvent the difficulty of lacking mono-specific antibodies against individual PRLs, stable NIH3T3 transfectants of the control vector or expression constructs of PRLs tagged with the Flag epitope were established. The transfectants were untreated or treated with PE at a dosage of 1 μg/ml or 10 μg/ml for 5 minutes, washed to remove cell-free drug and lysed for immunoprecipitation assays using an anti-Flag monoclonal antibody. SDS-PAGE/Western blotting showed that a Flag-tagged protein of approximately 23 kDa, as expected for Flag-PRL-1 was detected in the immunocomplexes from the Flag-PRL-1 transfectant (FIG. 3B, lane 4) but not in those from the vector control cells (FIG. 3B, lane 1). The immunocomplexes from the Flag-PRL-1 transfectant showed significant activity in dephosphorylating a synthetic phosphotyrosine peptide in PTPase assays (FIG. 3A, lane 4) whereas those from the vector control cells lacked such an activity (FIG. 3A, lane 1) demonstrating that the Flag-PRL-1 protein from the transfectant was an active PTPase. The immunocomplexes from the PE-treated Flag-PRL-1 transfectant failed to dephosphorylate the substrate (FIG. 3A, lanes 5 and 6) although they contained Flag-PRL-1 protein at levels similar to those from the untreated cells (FIG. 3B, lanes 5 and 6). Immunocomplexes from PE-treated Flag-PRL-2 (FIG. 3C) or Flag-PRL-3 (FIG. 3E) also lacked PTPase activity in comparison to those of the untreated transfectants despite approximately equal amounts of Flag-tagged PRLs in the samples (FIGS. 3D and 3F). These results demonstrated that the intracellular PRLs in the PE-treated transfectants were inactivated and that pentamidine was an effective inhibitor against PRLs in the transfectants.

### Example 4

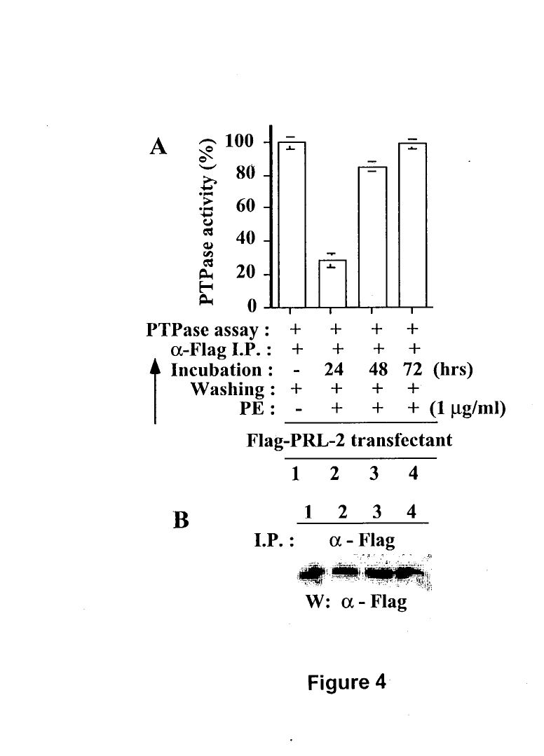

To assess the duration of pentamidine-induced inaction of intracellular PRLs, the activities of PRL-2 in NIH3T3 transfectants that were incubated for 24-72 hours following a brief treatment with 1 μg/ml pentamidine were measured. In FIG. 4A, PRL2 immunoprecipitated from transfectants treated with pentamidine for 5 minutes, washed to move cell-free drug and then incubated for 24 hours in drug-free medium showed relative PTPase activity of only 24% in comparison to that from the untreated cells. PRL-2 from cells incubated for 48 and 72 hours following pentamidine treatment showed relative PTPase activities of 86% and 98% respectively. In FIG. 4B, the amounts of PRL-2 protein in the immunocomplexes from the treated or untreated cells were comparable as determined by SDS-PAGE/Western blotting. Thus brief pentamidine-treatment had an inhibitory effect on the PTPase activity of intracellular PRL-2 that lasted at least for 24 hours and required more than 48 hours for its complete removal. Data represent mean±s.d. values of triplicate samples.

These results together demonstrate that pentamidine is a potent inhibitor of the PRLs and illustrate the anti-cancer activity of the drug via inactivating these oncogenic phosphatases.

### Example 5

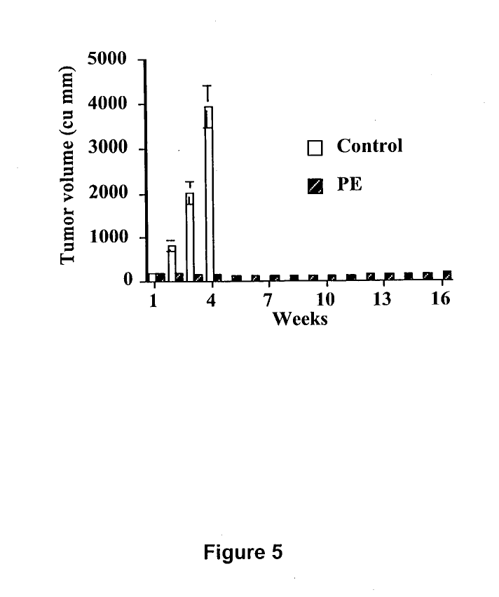

Pentamidine inhibits the growth of WM9 human melanoma tumors in nude mice. To assess the potential anti-cancer activity of pentamidine in vivo, the effects of the drug on the growth of nude mice tumors of WM9 human melanoma cell line, which express PRL-1, PRL-2, and PRL-3 were determined, as illustrated in FIG. 5.

WM9 cells inoculated in nude mice formed aggressively growing tumors that were markedly inhibited by pentamidine treatment (250 μg/mouse, every two days). During the 16 week-study period, the tumors in pentamidine-treated mice stayed at sizes similar to those at the treatment initiation point while the tumors in the control mice, subjected to no treatment, grew so rapidly that humane sacrifice of the animals was required at the fourth week. Data represent mean±SE (n=8).

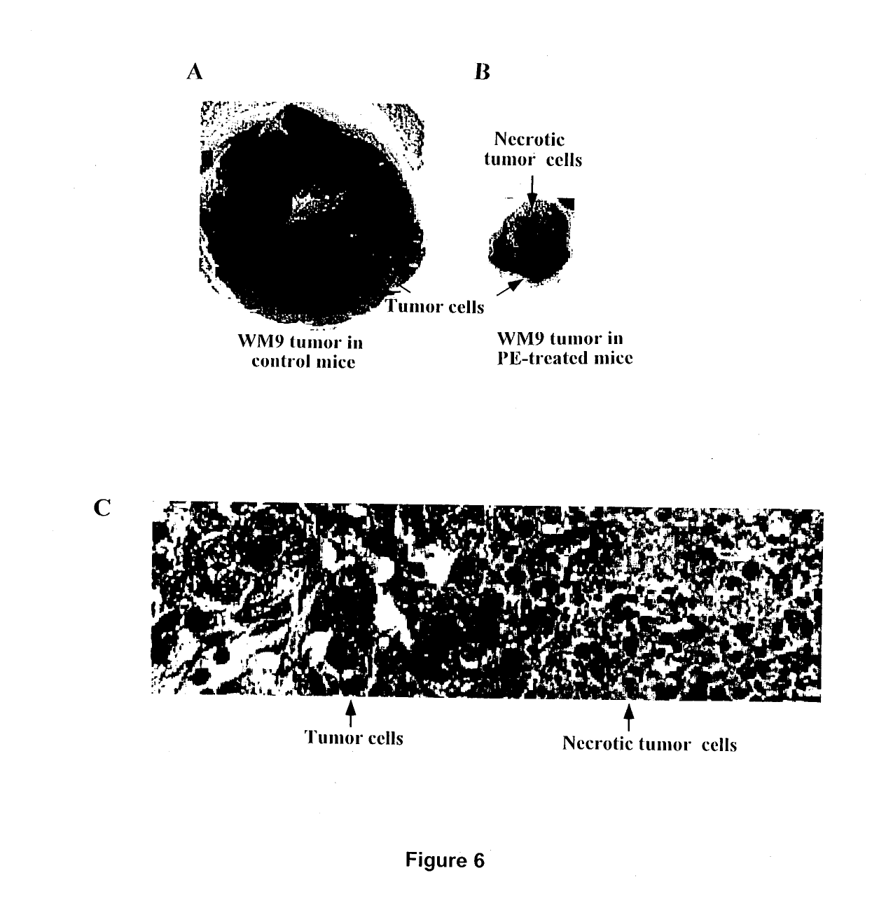

This pentamidine treatment caused no obvious abnormalities in the mice, which all survived and showed steady body weight gains during the study period. The internal organs (heart, kidney, liver, lung and spleen) of two pentamidine-treated mice subjected to histologic evaluation at the end of the study period were unremarkable. FIG. 6A is a representative view at 1× magnification of H.E.-stained sections of WM9 tumors in nude mice without treatment at the fourth week following inoculation. FIG. 6B is a representative view at 1× magnification of H.E.-stained sections of WM9 tumors in nude mice treated with pentamidine for 16 weeks. FIG. 6C is a higher power view (40×) of the sample in FIG. 6B. Tumors in these mice showed significant necrosis that accounted for more than 50% of the tumor mass as depicted in FIG. 6.

These results demonstrate a marked growth inhibitory activity of pentamidine at a non-toxic dose against WM9 tumors in nude mice that was characterized by extensive tumor cell necrosis.

### Example 6

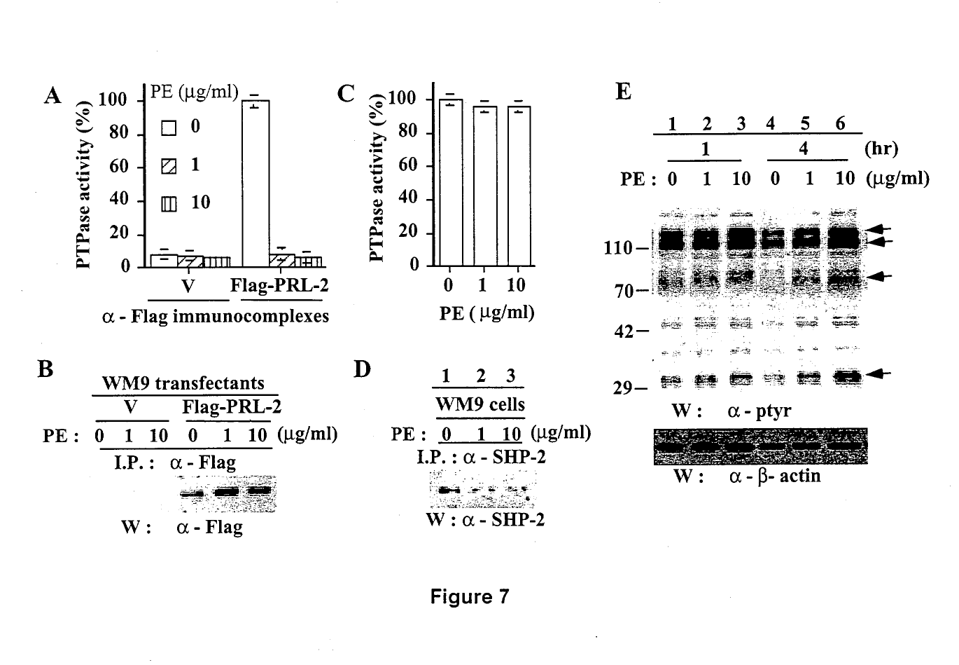

Pentamidine augments cellular protein tyrosine phosphorylation and inhibits PRL-2 in WM9 cells. PTPase inhibition by pentamidine in cancer cells was analyzed, as illustrated in FIG. 7. WM9 cells were transfected with an expression construct of Flag-PRL-2 or the control vector were untreated or treated with PE for 5 minutes, washed and lysed for immunoprecipitation assays using an anti-Flag antibody. A Flag-tagged protein of approximately 23 kDa, as expected for Flag-PRL-2, was detected in the immunocomplexes from Flag-PRL-2 transfectant but not in those from the vector control cells (FIG. 7A). The immunocomplexes from the untreated Flag-PRL-2 transfectant showed significant activity in dephosphorylating the phosphotyrosine peptide substrate while those from PE-treated Flag-PRL-2 transfectant showed activity similar to those of the vector control cells (FIG. 7B), demonstrating inactivation of the phosphatase in PE-treated WM9 cells. In contrast, SHP-2 immunoprecipitated from untreated or PE treated of WM9 cells had comparable PTPase activities (FIGS. 7C and 7D), indicating that this phosphatase in WM9 cells was insensitive to inhibition by PE, consistent with the insensitive nature of SHP-2 to the inhibitor in vitro (FIG. 1E). Treatment of WM9 cells with PE for 5 minutes resulted in increased tyrosine phosphorylation in several cellular proteins yet to be identified (FIG. 7E), consistent with inhibition of PTPases in the cancer cells by the drug.

These results demonstrated that PE functioned as an inhibitor that acted selectively against intracellular PRL-2 but not SHP-2 and induced cellular protein tyrosine phosphorylation in WM9 melanoma cells.

### Example 7

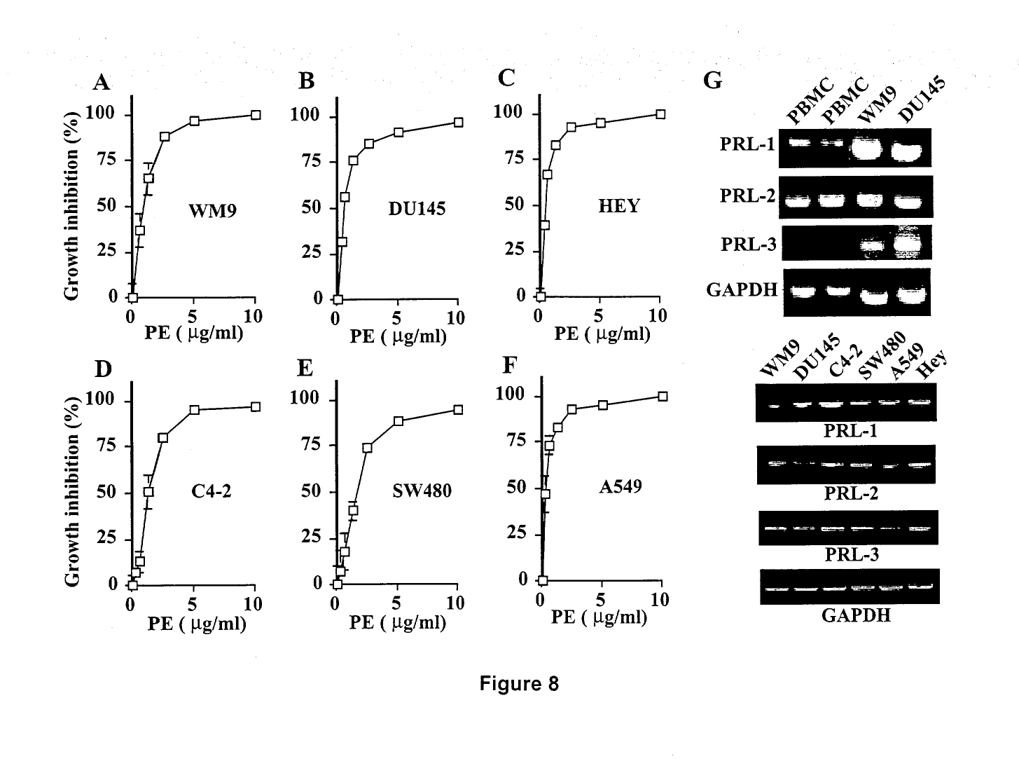

Pentamidine inhibits the in vitro growth of different human cancer cell lines that express PRLs. The effects of pentamidine on the in vitro growth of cell lines of different human malignancies, including prostate carcinoma (DU145 and C4-2) (FIGS. 8B and 8D), ovarian carcinoma (Hey) (FIG. 8C), colon carcinoma (SW480) (FIG. 8E) and lung carcinoma (A549) (FIG. 8F) was compared to growth in WM9 cells (melanoma) (FIG. 8A) for 6 days as determined by MTT assays. (Data represent mean±s.d. values of triplicate samples).

The growth of all six cell lines in cultured was inhibited by pentamidine in a dose-dependent manner with complete growth inhibition of the cell lines occurred at 10 μg/ml as confirmed by the absence of viable cells under microscopic examination. The drug also showed significant growth inhibitory effects at lower doses (0.3-5 μg/ml), close to its therapeutic dosage (2-4 mg/kg). Among the cell lines, A549 cells were most sensitive to the drug with 45% and 94% growth inhibition achieved at 0.3 μg/ml and 2.5 μg/ml respectively (FIG. 8C). Although the most resistant cell line SW480 was barely affected by the drug at 0.3 μg/ml, the growth of the cell line was significantly inhibited (74%) by pentamidine at 2.5 μg/ml (FIG. 8E). The other cell lines showed pentamidine-sensitivities falling between those of A549 and SW480 (FIGS. 8B, 8C and 8D). These results demonstrated a growth inhibitory activity of pentamidine against different human cancer cell lines. RT-PCR analysis revealed the presence of the transcripts of the PRLs in the six cell lines with PRL-1 and PRL-3 expression at levels higher than those in the peripheral blood mononuclear cells (PBMC) of two healthy volunteers (FIG. 8G). The control is glyceraldehyde-3-phosphate-dehydrogenase (GAPDH).

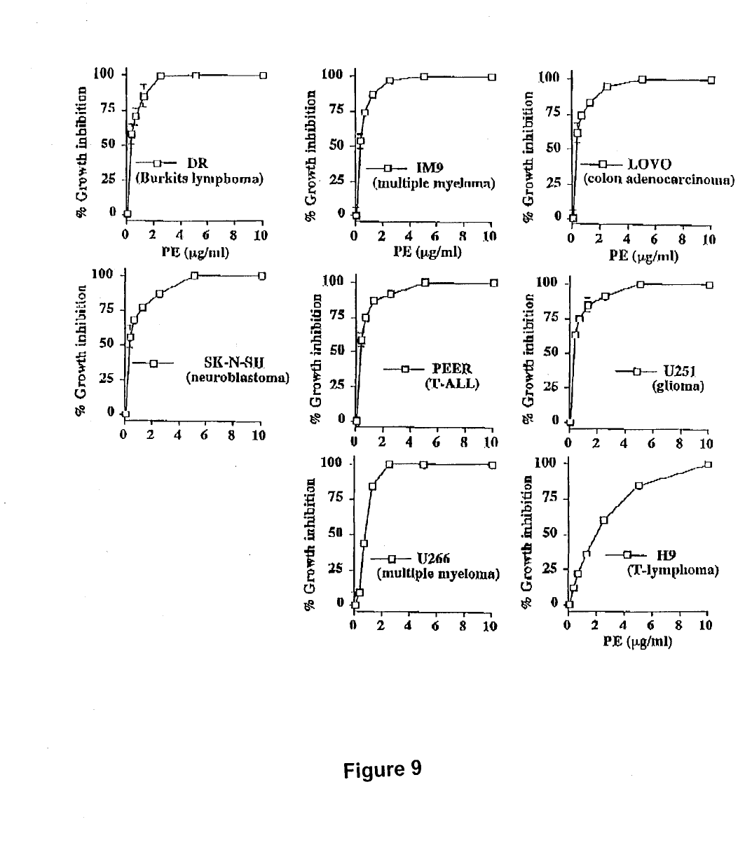

The growth inhibition of other cancer cell lines, such as Burkitts lymphoma (FIG. 9A), multiple myeloma (IM9 cells) (FIG. 9B), colon adenocarcinoma (LOVO cells) (FIG. 9C), neuroblastoma (SK—N—SH cells) (FIG. 9D), T-ALL (PEER cells) (FIG. 9E), glioma (U251 cells) (FIG. 9F), multiple myeloma (U266 cells) (FIG. 9G) and T-lymphoma (H9 cells) (FIG. 9H), with pentamidine in a dose-dependent manner was also investigated. Growth of cell lines of different human malignancies cultured in the absence or presence of various amounts of pentamidine for 6 days was determined by MTT assays. Data represent mean±s.d. values of triplicate samples. Again, complete growth inhibition of the cell lines occurred by 10 μg/ml. Complete inhibition occurred at or near therapeutic dosage (2-4 mg/kg) of pentamidine in DR cells, IM9 cells, LOVO cells, U251 cells and U266 cells.

### Example 8

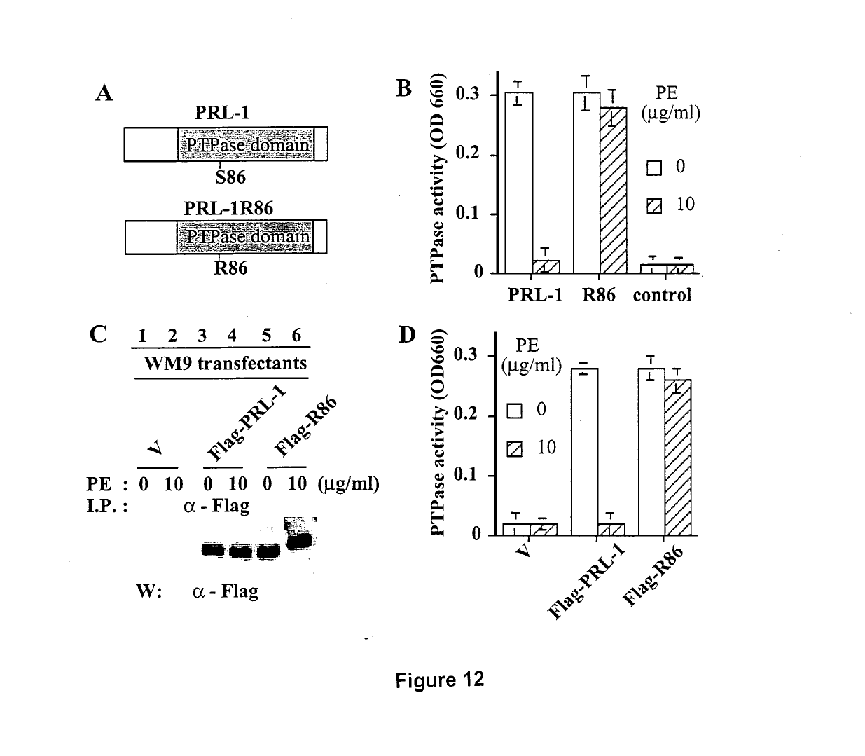

Intracellular PRL-1R86 was insensitive to pentamidine inhibition. To define the role of PRLs in the anti-cancer mechanism of pentamidine, the effects of pentamidine on the PTPase activity of a mutant PRL-1R86 and on the capacity of mutant PRL-1R86 to confer resistance to pentamidine-induced growth inhibition was determined. As shown in FIG. 12A, PRL-1R86 is a PRL-1 mutant, which contains a single amino acid residue substitution of a serine to arginine at position 86 in the PTPase domain. In FIG. 12B, in contrast to recombinant PRL-1 whose PTPase activity was inhibited by pentamidine comparable to control vector, recombinant PRL-1R86 had comparable PTPase activity that was not significantly reduced in the presence of pentamidine. Each reaction contained 10 ng of PRL-1 or PRL-1R86. Data represent mean±s.d. of triplicate samples. To assess the sensitivity of intracellular PRL-1R86 to pentamidine inhibition, stable WM9 transfectants of control vector (pBabepuro) or expression constructs of Flag-tagged PRL-1 or PRL-1R86 were generated. As illustrated in FIG. 12D, Flag-tagged PRL-1R86 immunoprecipitated from WM9 transfectant treated with pentamidine showed PTPase activity comparable to the ones from untreated cells whereas Flag-tagged PRL-1 from pentamidine treated cells had little PTPase activity. Relative amounts of Flag-tagged PRL-1 (Flag-PRL-1) or PRL-1R86 (Flag-R86) in immunocomplexes from WM9 transfectants untreated (0) or treated with pentamidine (5 min.) as determined by Western blotting are illustrated in FIG. 12C. Since similar amounts of Flag-tagged PRL-1 or PRL-1R86 proteins were present in the immunocomplexes, these differential effects of pentamidine treatment on PTPase activities of PRL-1 and PRL-1R86 demonstrated that intracellular PRL-1R86 was insensitive to pentamidine inhibition. Data represent mean±s.d. of triplicate samples.

### Example 9

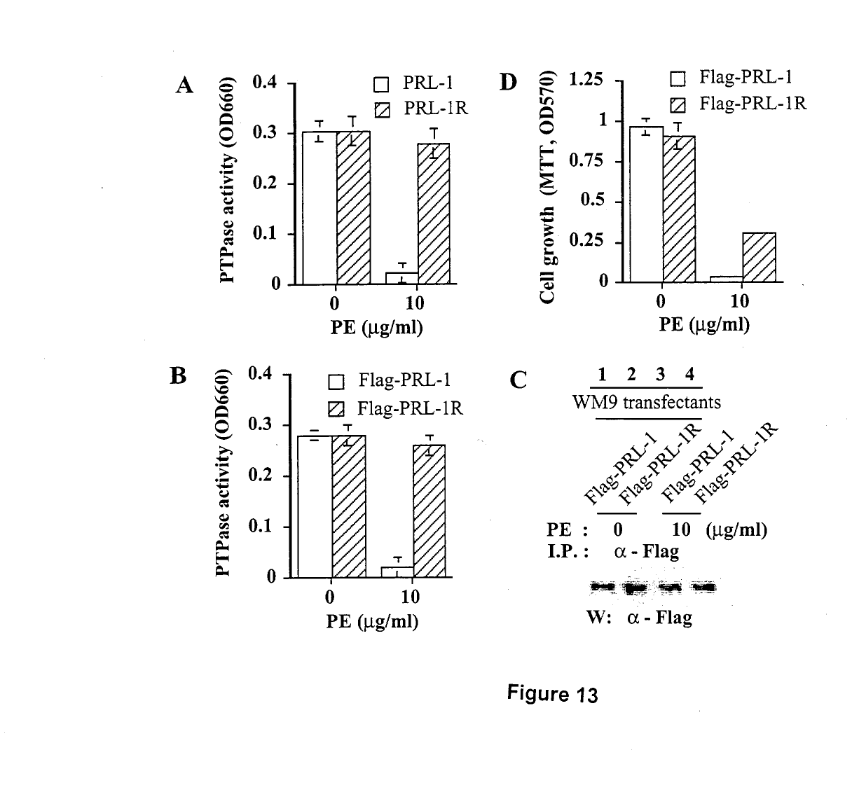

FIG. 13 illustrates that PE-insensitive PRL-1R86 confers resistance to PE-induced growth inhibition in WM9 melanoma cells. Stable WM9 transfectants of expression constructs of employing Flag-tagged PRL-1 or PRL-1R86 were generated. For measurement of pentamidine effects on cell growth in vitro, cells were cultured in the absence (−) or presence (+) of various amounts of pentamidine for 6 days with viable cells quantified by MTT assays as described. A comparison of assays of the PRL-1R86 and the PRL-1 activities in the WM9 cells confirmed these findings. A. recombinant proteins PTPase assays. B immunocomplex PTPase assays. C. immunocomplex Western blotting. D. Cell growth assays (MTT). Data represent mean±s.d. of triplicate samples. These results illustrate that PRL-1 and other PRLs are key targets for mediating PE anti-cancer activity.

### Example 10

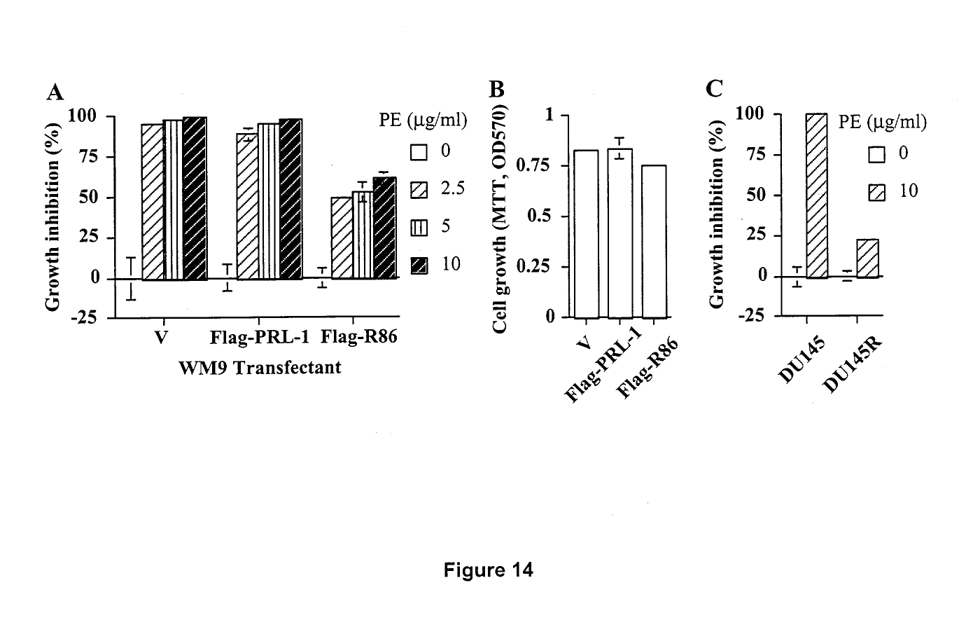

Ectopic expression of PRL-1R86 confers partial resistance to PE-induced growth inhibition in WM9 melanoma cells. The effects of pentamidine on the growth of the stable WM9 transfectants in culture was measured, as illustrated in FIG. 14. Pentamidine-induced growth inhibition of WM9 transfectants of control vector (V), Flag-PRL-1 or Flag-PRL-1R86 in day 6 culture was determined by MTT assays. Like the parental WM9 cells pentamidine at 2.5-10 μg/ml, nearly completely killed or completely killed transfectants of the vector (V) (1410) and Flag-PRL-1 (1420) (FIG. 14A). In contrast, the growth of PRL-1R86 transfectant was only partially inhibited (˜50%) by pentamidine under comparable conditions (FIG. 14A). FIG. 14B illustrates that in vitro growth rates of the transfectants in the absence of pentamidine were similar in day 6 culture. These results demonstrate that PRL-1R86 conferred partial resistance to pentamidine-induced growth inhibition in WM9 cells, suggesting that pentamidine growth inhibitory activity against cancer cells is mediated at least in part through inhibiting PRL-1. In support of this, pentamidine was found to have only a partial growth inhibitory effect against DU145R cells, in which the PRL-1R86 mutant is also expressed (FIG. 14C). Data represent mean±s.d. of triplicate samples.

### Example 11

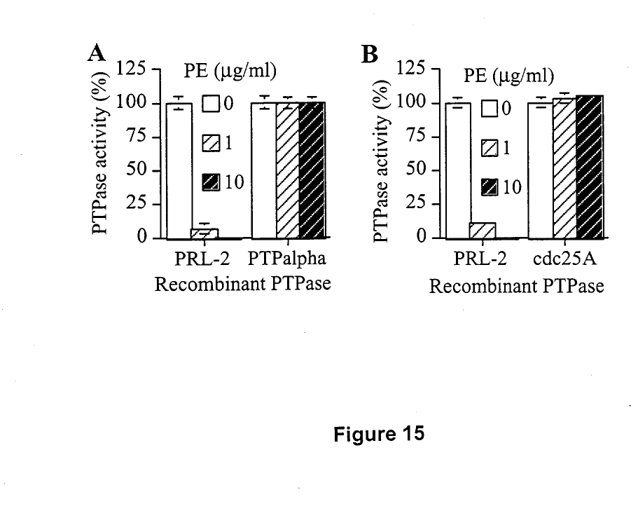

PE does not inhibit the PTPase activity of recombinant PTPalpha and cdc25A. To address whether PTPalpha and cdc25, oncogenic PTPases, also present in WM9 cells, are candidates in mediating PE-induced growth inhibition, the effects of PE on the activity of these PTPases was determined. A GST fusion protein of PTPalpha was obtained from Dr. J. den Hertog. In contrast to GST fusion protein of PRL-2 that was inhibited by PE, recombinant PTPalpha showed PTPase activity not affected in the presence of PE (FIG. 15A). Similarly, recombinant cdc25A (Upstate Group, Inc.)was insensitive to inhibition by PE, although PRL-2 was inhibited by the drug under comparable conditions (FIG. 15B).

These results demonstrated that PE had no inhibitory activity against recombinant PTPalpha and cdc25A, suggesting that these oncogenic PTPases are not PE targets and are unlikely to be involved in mediating PE anti-cancer effects. The observation that PE lacked inhibitory activity against PTPalpha and cdc25A also provides additional evidence that PE acts against only selective PTPases.

### Example 12

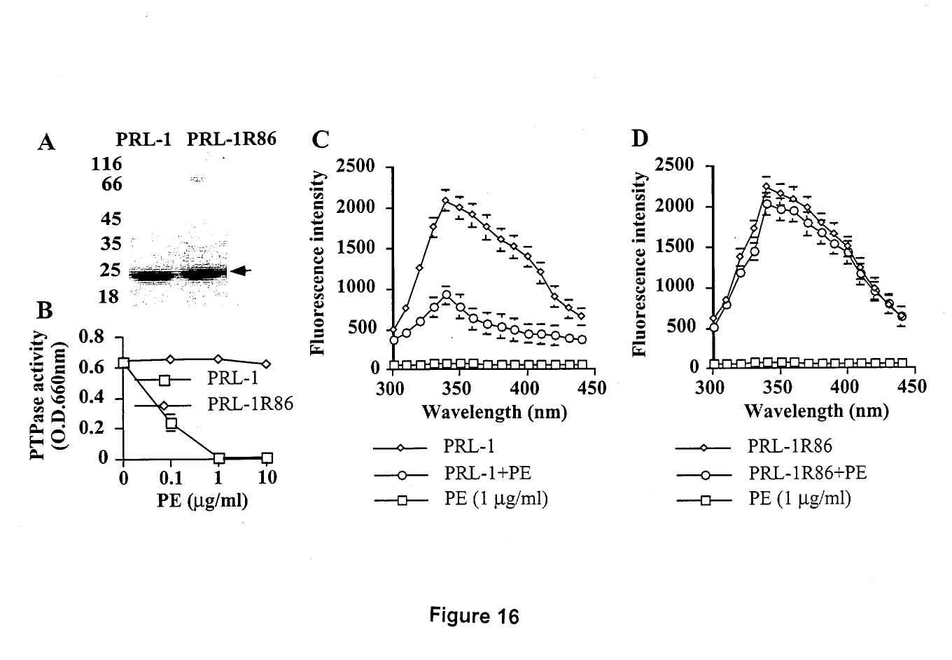

PE selectively quenches the intrinsic fluorescence of recombinant PRL-1 but not PRL-1R86 mutant. His-tagged PRL-1 and PRL-1R86 proteins were purified from bacteria transformed with pET16b constructs containing cDNAs encoding human PRL-1 or the mutant, respectively (FIG. 16A). Like GST fusion protein counterparts, his-PRL-1 and PRL-1R86 showed comparable PTPase activities, although PRL-R86 was insensitive to inhibition by PE (FIG. 16B). Both proteins showed intrinsic fluorescence with similar intensity profiles (FIGS. 16C and 16D). Intrinsic fluorescence of PRL-1 was quenched in the presence of PE, which showed little effect on the fluorescence of PRL-1R86 (FIGS. 16C and 16D).

Since intrinsic fluorescence is emitted by tryptophan residues in a protein and could be quenched when the protein forms a complex with another molecule that masks the tryptophan fluorescence, the results are consistent with a stable binding of PE to PRL-1. In this regard, the observation that PE failed to quench the fluorescence of PRL-1R86 insensitive to PE inhibition is significant. It further indicates that the stable binding of PE to PRL-1 is required for PE inhibition of the phosphatase. Without being bound by theory, it is believed that this binding provides a mechanistic explanation for the insensitivity of PRL-1R86 to PE inhibition, indicating that it resulted from abolishing such a binding due to the serine/arginine substitution. Moreover, it identifies serine 86 in PRL-1 as a key residue essential in PE/PRL-1 interaction and inhibition mechanism.

### Example 13

PE-related chemical PR lacks inhibitory activity and quenching capacity against PRL-1. PR (propamidine) is a synthetic chemical structurally similar to PE. A comparison of the structures of PE and PR is shown in FIG. 17A. PR was obtained from Rhone-Poulenc in the form of propamidine isethionate, comparable to the form of PE used in our studies (pentamidine isethionate). PR had little effect on recombinant his-PRL-1, which was inhibited by PE under comparable conditions (FIG. 17B), and failed to quench the intrinsic fluorescence of the protein (FIG. 17C). PR also failed to inhibit recombinant PRL-2 and PRL-3 (data not shown).

These results demonstrated that shortening the linker in PE from (CH₂)₅ to (CH₂)₃ abolished PE's inhibitory activity and quenching capacity against PRL-1, indicating the (CH₂)₅ linker as a key element in PE/PRL-1 interaction and PE inhibition of the phosphatase. Moreover, the fact that PR lacked both inhibitory activity and quenching capacity provides additional evidence that inhibition mechanism is mediated via PE binding to its target phosphatases. These observations together support that hypothesis that PE inhibits PRLs via inter-molecular interaction requiring a specific chemical substructure in PE (e.g., the linker in PE) and unique residues in the phosphatases (e.g, Serine 86 of PRL-1).

### Example 14

PE augments IFNα-induced growth inhibition and Stat1 phosphorylation in WM9 cells. A putative mode of action of PE against leishmaniasis is via targeting PTP1B to augment leishmaniacidal activity of host cytokines. Without being bound by theory, this was proposed based on our previous observations of an inhibitory activity of PE against PTP1B and the negative regulatory role of the phosphatase in the signaling of cytokines with leishmaniacidal activity. This hypothesis predicts that PE might augment intracellular signaling and growth inhibitory effects of IFNs against cancer cells. As an initial step to test the hypothesis, we determined the growth inhibitory effects of PE/IFNα combination in comparison to individual drugs against WM9 cells. Tyrosine phosphorylation levels of Stat1 in WM9 cells were quantified as an indicator of IFNα signaling in the cancer cells.

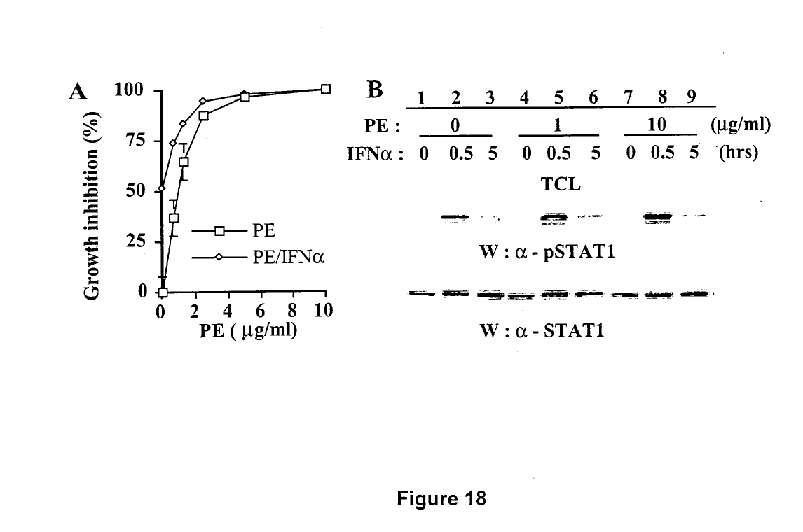

The growth of WM9 cells in culture was partially inhibited by IFNα (1000 U/ml) and by PE (0.6-2.5 μg/ml as single agents (FIG. 18A). Growth inhibition was increased with combinations of the two drugs at these dose ranges (FIG. 18A), indicating a positive interaction between drugs against the cancer cells. Such an interaction was undetectable when IFNα was combined with PE at higher doses (5-10 μg/ml), which by itself induced complete killing of the cancer cells (FIG. 18A). IFNα-induced stat1 phosphorylation in WM9 cells was increased in the presence of PE (FIG. 18B, comparing lanes 5 and 8 to lane 1). Expression of PTP1B in WM9 cells was detected by gene expression profile analysis (our unpublished results) and by western blotting (data not shown) although whether intracellular PTP1B in WM9 cells is sensitive to inhibition by PE treatment has not been determined.

These results support a putative action of PE via targeting PTP1B to augment cytokine signaling and anti-leishmania effects and suggest that PE might be beneficial in combination with IFNs against cancer cells. Significantly, the observation that PE alone failed to induce Stat1 phosphorylation (FIG. 18, lanes 4 and 7) suggests that growth inhibition of the WM9 cells by PE as a single agent was not a result of activating Stat1 through targeting PTP1B. It is consistent with the hypothesis that PE has direct anti-cancer activity via blocking the oncogenic activity of PRLs.

### Example 15

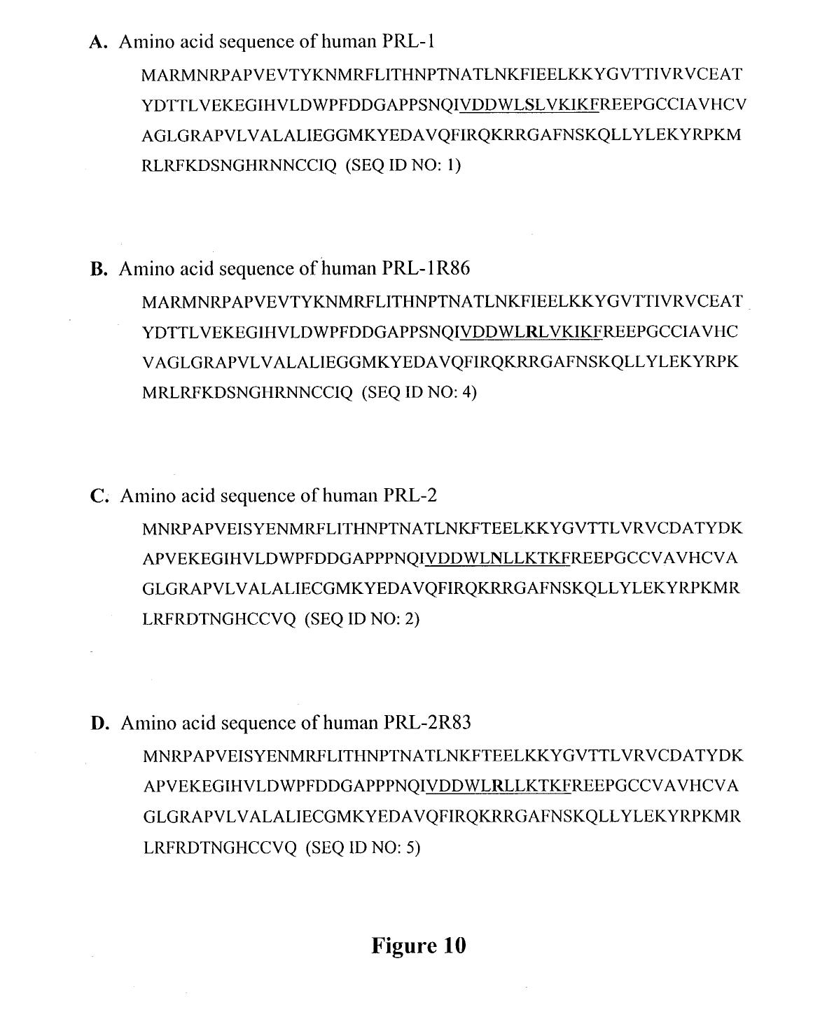

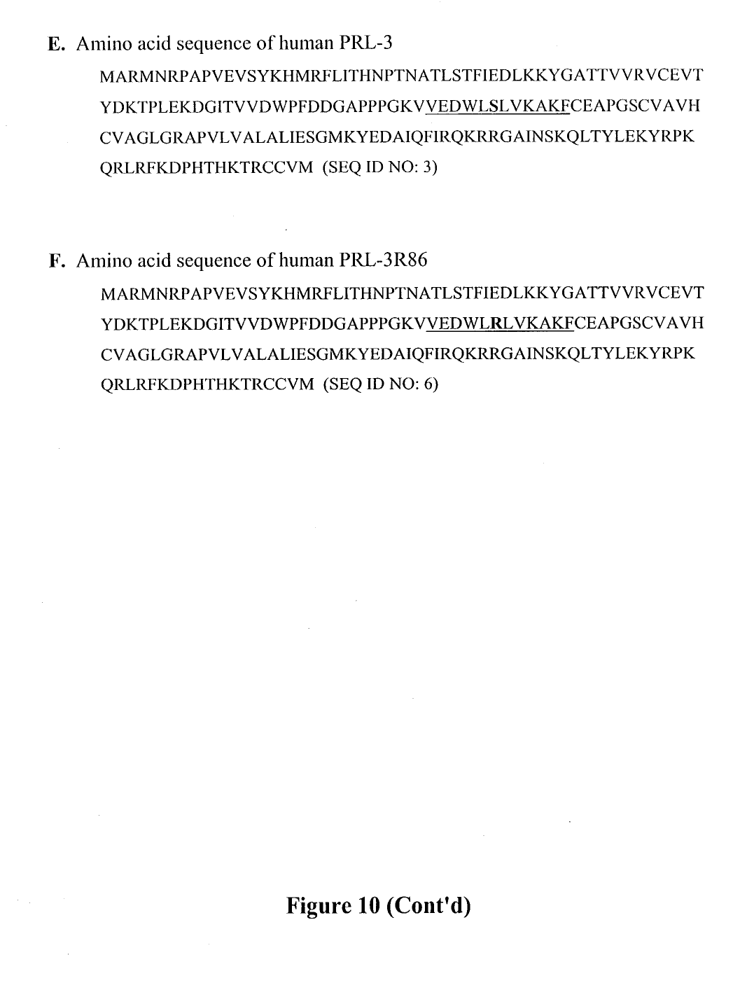

Pentamidine inhibition of the three PRLs is mediated via common mechanism based on pentamidine binding to a conserved sub-domain in the phosphatases: identification of the PRL-1 serine 86 counterpart residues in PRL-2 and PRL-3 required for PE inhibition and development of PE-insensitive PRL-2 and PRL-3 mutants. The amino acid sequences of PRL-1 (SEQ ID NO: 1), PRL-2 (SEQ ID NO: 2) and PRL-3 (SEQ ID NO: 3) are illustrated in FIGS. 10A, C and E, respectively. Without being bound by theory, we hypothesized that PE inhibition of the three PRLs might be mediated via a common mechanism based on PE binding to a conserved sub-domain in the phosphatases. This is supported by the observation that PE showed similar activity against all three PRLs, which have ˜70% homology in amino acid residues. Given that PRL-1 serine at position 86 is required for PE/PRL-1 interaction and PE inhibition of the phosphatase, and that its substitution by an arginine resulted in a PE-insensitive PRL-1R86 (FIG. 10B, SEQ ID NO: 4), the hypothesis predicts that the S86 counterpart residues in the other two PRLs might play a similar role. Thus substitution of the counterpart residue could result in PE-insensitive mutants of PRL-2 and PRL-3.

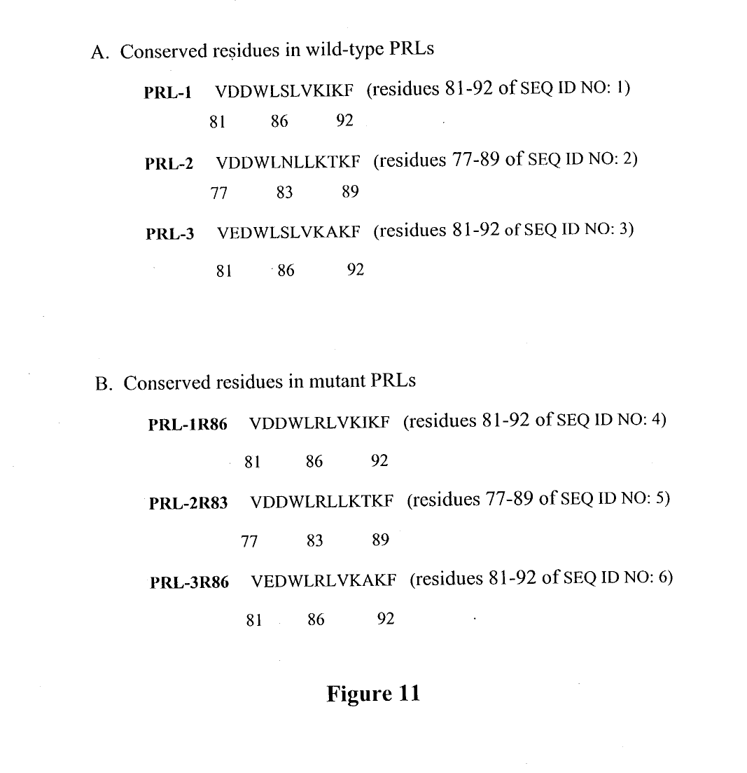

To test this hypothesis, we identified N83 of PRL-2 and S86 of PRL-3 as potential PRL-1R86 counterparts in the phosphatases based on an amino acid sequence motif near PRL-1S86 and conserved in all three PRLs. The two amino acids are both non-ionic polar residues and might be chemically similar in terms of H−=bonding/ionic pairing. cDNAs encoding PRL-2R83 and PRL-3R86 were generated through introducing single nucleotide changes in the cDNAs of wild type PRLs via recombinant DNA technology following established procedures (Jiao, H., et al. (1996) Mol. Cell. Biol. 16, 6985-6992), cloned into pGEX vector and introduced into bacteria. GST fusion proteins of PRL-2R83 and PRL-3R86 were purified and characterized. The amino acid sequences of the mutants PRL-2R83 and PRL-3R86 are illustrated in FIGS. 10D and 10F, SEQ ID NO: 5 and SEQ ID NO: 6, respectively. The conserved residues in the wild-type PRLs are illustrated in FIG. 11A and the conserved residues in mutant PRLs are illustrated in FIG. 11B.

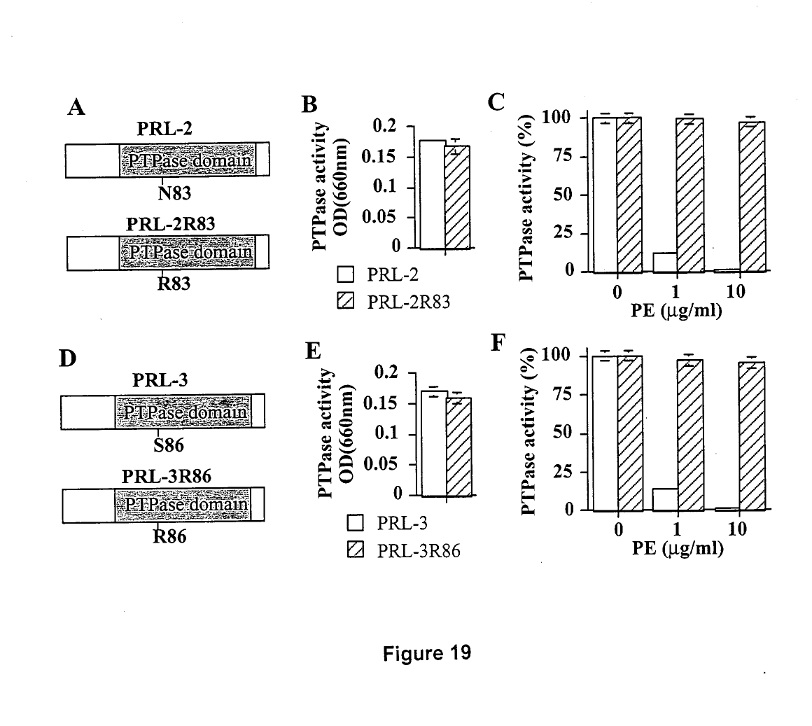

The structures of PRL-2 and PRL-2R83 are illustrated in FIG. 19A, and the structures of PRL-3 and PRL-3R86 are illustrated in FIG. 19D. FIG. 19B shows the PTPase activities of GST (control) or GST fusion proteins (10 ng/reaction) of PRL-2 or PRL-2R83 as determined by PTPase assays using a phosphotyrosine peptide substrate. FIG. 19C illustrates relative PTPase activities of PRL-2 and PRL-2R83 in the absence or presence of pentamidine. FIG. 19E shows the PTPase activities of GST (control) or GST fusion proteins (10 ng/reaction) of PRL-3 or PRL-3R86 as determined by using the peptide substrate. Finally, FIG. 19F shows the relative PTPase activities of PRL-3 and PRL-3R86 in the absence or presence of pentamidine. Data represent mean±s.d. of triplicate samples.

As illustrated in FIG. 19B, PRL-2R83 showed PTPase activity comparable to that of PRL-2 but was not inhibited in the presence of PE (FIG. 19C). Similarly, PRL-3R86 also had PTPase activity (FIG. 19E) that was insensitive to PE inhibition (FIG. 19F). These results demonstrated that PRL-2 N83 and PRL-3 S86 are the PRL-1 S86 counterpart residues required for PE inhibition of the two PTPases in vitro. Their identification provides direct evidence supporting our hypothesis that PE inhibits the PRLs via binding to a sub-domain conserved in the PTPases.

Significantly, the development of the PE-insensitive PRLs allows us to assess the roles of individual PRLs in PE anti-cancer effects through determining their capacities to confer resistance to PE-induced growth inhibition in cancer cells.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have elements that do not differ from the literal language of the claims, or if they include equivalent elements with insubstantial differences from the literal language of the claims.

I claim:

1. A therapeutic composition for preventing, treating or ameliorating cancer, comprising pentamidine, or a biological equivalent or derivative thereof.

2. The composition of claim 1, wherein the pentamidine, or the biological equivalent or derivative thereof, is present in an amount effective to inhibit phosphatase activity in cancer cells.

3. The composition of claim 2, wherein the effective amount is a clinically tolerated dosage.

4. The composition of claim 2, wherein the effective amount is about 2 to about 4 mg/kg.

5. The composition of claim 2, wherein the phosphatase is a PRL phosphatase.

6. The composition of claim 5, wherein the PRL phosphatase is selected from the group consisting of PRL-1, PRL-2, PRL-3, and combinations thereof.

7. The composition of claim 1, wherein the cancer is a human cancer.

8. The composition of claim 1, wherein the cancer is selected from the group consisting of lymphoma, multiple myeloma, colon cancer, neuroblastoma, glioma, leukemia, melanoma, prostate cancer, breast cancer, renal cancer, bladder cancer, and combinations thereof.

9. A method for preventing, treating or ameliorating cancer, comprising administering to a mammal pentamidine, or a biological equivalent or derivative thereof.

10. The method of claim 9, wherein the mammal is a human.

11. The method of claim 9, wherein the pentamidine, or a biological equivalent or derivative thereof, is administered in an amount effective to inhibit phosphatase activity in cancer cells.

12. The method of claim 11, wherein the effective amount is a clinically tolerated dosage.

13. The method of claim 11, wherein the effective amount is about 2 to about 4 mg/kg.

14. The method of claim 9, wherein the pentamidine, or the biological equivalent or derivative thereof, is administered in an amount effective to inhibit the activity of a PRL phosphatase in cancer cells.

15. The method of claim 14, wherein the PRL phosphatase is selected from the group consisting of PRL-1, PRL-2, PRL-3, and combinations thereof.

16. The method of claim 9, wherein the cancer is a human cancer.

17. The method of claim 9, wherein the cancer is selected from the group consisting of lymphoma, multiple myeloma, colon cancer, neuroblastoma, glioma, leukemia, melanoma, prostate cancer, breast cancer, renal cancer, bladder cancer, and combinations thereof.

18. A method for inhibiting phosphatase activity in cancer cells, comprising administering to the cancer cells an effective amount of pentamidine, or a biological equivalent or derivative thereof.

19. The method of claim 18, wherein the effective amount is a clinically tolerated dosage.

20. The method of claim 18, wherein the effective amount is about 2 to about 4 mg/kg.

21. The method of claim 18, wherein the amount of pentamidine, or the biological equivalent or the derivative thereof, is effective to inhibit activity of a PRL phosphatase.

22. The method of claim 21, wherein the PRL phosphatase is selected from the group consisting of PRL-1, PRL-2, PRL-3, and combinations thereof.

23. A method for identifying pentamidine-resistant or pentamidine-sensitive cancer cells, comprising:
(a) isolating a PRL phosphatase from a cancer cell sample; and
(b) determining an amino acid sequence of the isolated PRL phosphatase, wherein the presence of a mutant amino acid sequence indicates pentamidine resistant cancer cells and the absence of a mutant amino acid sequence indicates pentamidine-sensitive cancer cells.

24. A method for determining a risk for pentamidine-resistance or pentamidine-sensitivity, comprising:
(a) isolating PRL phosphatase from a cancer cell sample; and
(b) determining an amino acid sequence of the isolated PRL phosphatase, wherein the presence of a mutant amino acid sequence indicates a risk for pentamidine resistance and the absence of a mutant amino acid sequence indicates pentamidine sensitivity.

25. A method for identifying pentamidine-resistant or pentamidine-sensitive cancer cells, comprising:
(a) isolating a PRL phosphatase from a cancer cell sample; and
(b) testing the isolated phosphatase for phosphatase activity in the presence and absence of pentamidine, or a biological equivalent or derivative thereof, wherein inhibition of the phosphatase activity is indicative of pentamidine-sensitive cancer cells, and lack of inhibition of the phosphatase activity is indicative of pentamidine-resistant cancer cells.

26. A method for identifying pentamidine-resistant or pentamidine-sensitive cancer cells, comprising:
(a) isolating a cancer cell sample; and
(b) performing a cell growth assay to determine the growth of the cancer cells in the presence and absence of pentamidine, or a biological equivalent or derivative thereof,
wherein inhibition of the cell growth is indicative of pentamidine-sensitive cancer cells, and lack of inhibition of the cell growth is indicative of pentamidine-resistant cancer cells.

27. A method for treating cancer, comprising administering an effective amount of a therapeutic composition comprising an agent that selectively inhibits a PRL phosphatase.

28. The method of claim 24, wherein the agent comprises pentamidine, or a biological equivalent or derivative thereof.

29. A polypeptide comprising SEQ ID NO: 4.

30. A polypeptide comprising SEQ ID NO: 5.

31. A polypeptide comprising SEQ ID NO: 6

32. A mutant PRL phosphatase produced by in vitro substitution of one or more amino acid residues of a wild-type PRL phosphatase.

33. The mutant PRL phosphatase of claim 32, wherein phosphatase activity of the mutant is not inhibited by pentamidine or a biological equivalent thereof.

34. The mutant PRL phosphatase of claim 32, wherein phosphatase activity of the mutant is not inhibited by a derivative of pentamidine.

35. A mutant PRL phosphatase comprising a substitution of one or more amino acid residues of a wild-type PRL phosphatase.

36. A method for inhibiting phosphatase activity in mammalian cells, comprising administering to the mammalian cells an effective amount of pentamidine, or a biological equivalent or derivative thereof.

37. The method of claim 36, wherein the amount of pentamidine, or the biological equivalent or the derivative thereof, is effective to inhibit activity of a PRL phosphatase.

38. The method of claim 37, wherein the PRL phosphatase is selected from the group consisting of PRL-1, PRL-2, PRL-3, and combinations thereof.

39. The method of claim 36, wherein the amount of pentamidine, or the biological equivalent or the derivative thereof, is effective to inhibit activity of a PTP1B phosphatase.

40. A therapeutic composition for preventing, treating or ameliorating a mammalian disease having an etiology related to cellular phosphatase activity, comprising pentamidine, or a biological equivalent or derivative thereof.

41. The composition of claim 40, wherein the pentamidine, or the biological equivalent or derivative thereof, is present in an amount effective to inhibit phosphatase activity in the cells.

42. The composition of claim 41, wherein the effective amount is a clinically tolerated dosage.

43. The composition of claim 40, wherein the phosphatase is a PTP1B phosphatase.

44. The composition of claim 40, wherein the phosphatase is a PRL phosphatase.

45. The composition of claim 44, wherein the PRL phosphatase is selected from the group consisting of PRL-1, PRL-2, PRL-3, and combinations thereof.

46. The composition of claim 40, wherein the disease is a human disease.

47. A method for preventing, treating or ameliorating a mammalian disease having an etiology related to cellular phosphatase activity, comprising administering to the mammal an effective amount of a therapeutic composition comprising pentamidine, or a biological equivalent or a derivative thereof.

48. The method of claim 47, wherein the pentamidine, or the biological equivalent or derivative thereof, is present in an amount effective to inhibit phosphatase activity in the cells.

49. The method of claim 48, wherein the effective amount is a clinically tolerated dosage.

50. The method of claim 48, wherein the effective amount is about 2 to about 4 mg/kg.

51. The method of claim 48, wherein the phosphatase is a PTP1B phosphatase.

52. The method of claim 48, wherein the phosphatase is a PRL phosphatase.

53. The method of claim 52, wherein the PRL phosphatase is selected from the group consisting of PRL-1, PRL-2, PRL-3, and combinations thereof.

54. The method of claim 48, wherein the disease is a human disease.


SEQUENCE LISTING (13 sequences)

SEQ ID NO: 1 - protein, 173 aa, Human
MARMNRPAPVEVTYKNMRFLITHNPTNATLNKFIEELKKYGVTTIVRVCEATYDTTLVEKEGIHVLDWPFDDGAPPSNQIVDDWLSLVKIKFREEPGCCIAVHCVAGLGRAPVLVALALIEGGMKYEDAVQFIRQKRRGAFNSKQLLYLEKYRPKMRLRFKDSNGHRNNCCIQ

SEQ ID NO: 2 - protein, 167 aa, Human
MNRPAPVEISYENMRFLITHNPTNATLNKFTEELKKYGVTTLVRVCDATYDKAPVEKEGIHVLDWPFDDGAPPPNQIVDDWLNLLKTKFREEPGCCVAVHCVAGLGRAPVLVALALIECGMKYEDAVQFIRQKRRGAFNSKQLLYLEKYRPKMRLRFRDTNGHCCVQ

SEQ ID NO: 3 - protein, 173 aa, Human
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRAPVLVALALIESGMKYEDAIQFIRQKRRGAINSKQLTYLEKYRPKQRLRFKDPHTHKTRCCVM

SEQ ID NO: 4 - protein, 173 aa, Artificial
  Modified human PRL-1.
MARMNRPAPVEVTYKNMRFLITHNPTNATLNKFIEELKKYGVTTIVRVCEATYDTTLVEKEGIHVLDWPFDDGAPPSNQIVDDWLRLVKIKFREEPGCCIAVHCVAGLGRAPVLVALALIEGGMKYEDAVQFIRQKRRGAFNSKQLLYLEKYRPKMRLRFKDSNGHRNNCCIQ

SEQ ID NO: 5 - protein, 167 aa, Artificial
  Modified human PRL-2.
MNRPAPVEISYENMRFLITHNPTNATLNKFTEELKKYGVTTLVRVCDATYDKAPVEKEGIHVLDWPFDDGAPPPNQIVDDWLRLLKTKFREEPGCCVAVHCVAGLGRAPVLVALALIECGMKYEDAVQFIRQKRRGAFNSKQLLYLEKYRPKMRLRFRDTNGHCCVQ

SEQ ID NO: 6 - protein, 173 aa, Artificial
  Modified human PRL-3.
MARMNRPAPVEVSYKHMRFLITHNPTNATLSTFIEDLKKYGATTVVRVCEVTYDKTPLEKDGITVVDWPFDDGAPPPGKVVEDWLRLVKAKFCEAPGSCVAVHCVAGLGRAPVLVALALIESGMKYEDAIQFIRQKRRGAINSKQLTYLEKYRPKQRLRFKDPHTHKTRCCVM

SEQ ID NO: 7 - protein, 13 aa, Artificial
  Synthetic phosphotyrosine peptide substrate for PTPase assays.
RRLIEDAEYAARG

SEQ ID NO: 8 - DNA, 32 bp, Human
  primer_bind (1)..(32): huPRL-3/5 Primer
taggatcccgggaggcgccatggctcggatga

SEQ ID NO: 9 - DNA, 32 bp, Human
  primer_bind (1)..(32): huPRL-3/3 Primer
gagtcgaccataacgcagcaccgggtcttgtg

SEQ ID NO: 10 - DNA, 33 bp, Human
  primer_bind (1)..(33): huPRL-2/5 Primer
taggatccccataatgaaccgtccagcccctgt

SEQ ID NO: 11 - DNA, 32 bp, Human
  primer_bind (1)..(32): huPRL-2/3 Primer
gagtcgacctgaacacagcaatgcccattggt

SEQ ID NO: 12 - DNA, 33 bp, Human
  primer_bind (1)..(33): huPRL-1/5 Primer
taggatccccaacatggctcgaatgaaccgccc

SEQ ID NO: 13 - DNA, 32 bp, Human
  primer_bind (1)..(32): huPRL-1/3 Primer
gagtcgacttgaatgcaacagttgtttctatg